(19) United States
(12) Patent Application Publication
(10) Pub. No.: US 2026/0294805 A1
(43) Pub. Date: Oct. 1, 2026

(54) PROGRAMMABLY DEGRADABLE HYDROGELS FOR FUNCTIONAL MOLECULAR RELEASE

(71) Applicant: GEORGIA TECH RESEARCH CORPORATION, Atlanta, GA (US)

(72) Inventors: M.G. FINN, Atlanta, GA (US); Wenting SHI, Atlanta, GA (US); Jimin HWANG, Atlanta, GA (US); Kasie COLLINS, Roswell, GA (US)

(21) Appl. No.: 19/476,958

(22) PCT Filed: Apr. 22, 2024
(86) PCT No.: PCT/US2024/025679

§ 371 Date: Oct. 20, 2025

Related U.S. Application Data
(60) Provisional application No. 63/497,430, filed on Apr. 20, 2023.

(51) Int. Cl. A61K 9/06; A61K 39/00; A61K 39/12; A61K 39/385; A61K 47/10; A61K 47/36; C08G 81/02

(52) U.S. Cl. CPC A61K 9/06; A61K 39/12; A61K 39/385; A61K 47/10; A61K 47/36; C08G 81/025; A61K 2039/5258; A61K 2039/6087; A61K 2039/62

(57) ABSTRACT

Disclosed herein are compositions including polymers functionalized with oxanorbornadiene (“OND”) crosslinkers and various within, where the materials and formulations thereof enable controlled, extended release of cargos such as therapeutic, antigenic, diagnostic, and/or analytical agents. Also disclosed is the use of physical entrapment of the cargo, covalent connection of the cargo to polymer via cleavable linkages, and combinations thereof. These combinations provide different patterns and durations of release of cargo molecules. Also disclosed herein are the methods of making and using the same.

### CROSS-REFERENCE TO RELATED APPLICATION

This application claims the benefit of U.S. Provisional Application 63/497,430, filed Apr. 20, 2023, the contents of which are hereby incorporated in its entirety.

### ACKNOWLEDGEMENT OF GOVERNMENT SUPPORT

This invention was made with government support under Grant No. GR10002313 awarded by the National Institutes of Health. The government has certain rights in the invention.

### BACKGROUND

Subunit vaccines have gained increased attention in recent years due to their reduced risk of side effects, ease of production, and enhanced stability. In addition to selecting the appropriate combination of antigens and adjuvants, a substantial body of research from the past decade has emphasized the importance of controlling vaccine kinetics, which refers to the temporal changes in immunogen concentration within the immune system, for augmenting overall vaccine efficacy. Specifically, sustained vaccine exposure can elicit more potent and long-lasting adaptive immune responses. This is achieved not only by reinforcing the interaction between immunogens and antigen-presenting cells (APCs), ensuring efficient antigen uptake, but also by enhancing the germinal center (response) cycles, which lead to the production of high-affinity antibodies.

Among various extended-release strategies, injectable hydrogels as vaccine depots are particularly appealing due to their unique advantages. These advantages include: 1) depot formations that better emulate natural viral infections by generating localized high antigen concentrations for extended periods, thus provoking a more potent immune response; 2) the ability to preserve the vaccine cargo's aqueous solvation and closely mimic the mechanical properties of adjacent tissues; and 3) injectability, allowing for minimally invasive cargo delivery. However, unlike microneedles or certain microparticle systems, fine-tuning the cargo release profile from a hydrogel platform remains challenging, and many injectable hydrogel depots suffer from uncontrolled initial burst releases. Consequently, the impact and potential benefits of various specific dosing profiles (such as initial-burst, constant, pulsatile, and exponentially increasing) on humoral immunization have not yet been investigated using hydrogel materials.

There remains a need for injectable hydrogel systems that allow for the tuning of gel degradation and programmable release of cargos such as therapeutic agents, especially biological therapeutic agents, including hormones, enzymes, toxins, antibodies, subunit vaccines, nucleoprotein complexes, other proteins, oligo- and polysaccharides, oligo- and polynucleotides, and other biomolecules.

### BRIEF DESCRIPTION OF THE FIGURES

FIG. 1 depicts the synthesis of various OND linkers.

FIG. 2 depicts the synthesis of dextran-PEG hydrogels with OND crosslinkers.

FIG. 3 depicts gelation kinetics from “fast” and “slow” gelation ONDs.

FIG. 4 depicts in vitro VLP release from hydrogels at 37° C. (repeat trials n=3).

FIG. 5 depicts synthesis of various OND linkers and OND-modified dextran. (a) OND 2-4 synthesis. (b) Hydrolysable dextran-OND synthesis. (c) Non-hydrolysable dextran-OND synthesis.

FIG. 6 depicts (a) Schematic representation of formation and degradation of dextran-OND/PEG-SH hydrogels. (b) OND linkages attached to dextran at the pendant carboxylic acid as shown in FIG. 1b. t½=the half-life of rDA reaction in CDCl3 at room temperature. (c) Gelation times measured for 10 wt % gels (1×PBS, pH 7.4 or 6.0, at room temperature). A few measurements were tested on ice. Data are provided as mean t standard deviation (n=3).

FIG. 7 depicts in vitro release profiles of VLPs and polystyrene beads from hydrogels at 37° C.

FIG. 8 depicts in vitro VLP released from non-hydrolysable hydrogels at 37° C. The ‘*’ symbol denotes dextran-OND connected via a non-hydrolysable triazole linkage. 2A represents OND with EWG=CO₂Et while 2a represents OND with EWG=CO₂Me.

FIG. 9 depicts in vitro mAb release profiles from hydrogels at 37° C.

FIG. 10 depicts the evaluation of in vivo formation of OND-hydrogel and release of dye-labeled mAb from OND-hydrogel compared to a bolus injection. (a) Schematic of the injection location for both bolus and depot groups, where both groups received a total of 200 μg of mAb in total. (b) Illustrated the experimental timeline where data was collected every three days, until termination at 8 weeks. (c) Images depicting the Cy5-labeled mAb cargo and the IR800CW-labeled hydrogel depot at the injection site on day 0. (d) No significant changes in mouse weight were observed. (e) Cargo concentration in sera demonstrated a higher blood concentration in the depot group compared to the bolus group.

FIG. 11 depicts (a) Schematic representation of formation and degradation of dextran-OND/PEG-SH hydrogels with nucleophile-functionalized cargo. (b) Validation of expressed and purified sfGFP. (c) Traut's mediated thiol functionalization of sfGFP. (d) In vitro sfGFP release profiles at 37° C. Black curve represents the entrapment method while the color curves represent release of covalently attached cargo.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes, from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds disclosed herein may be provided in the form of physiologically acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, naphthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions, Wiley Interscience, New York, 1981; Wilen et al., Tetrahedron 33:2725 (1977); Eliel, E. L. Stereochemistry of Carbon Compounds, McGraw-Hill, NY, 1962; and Wilen, S. H., Tables of Resolving Agents and Optical Resolutions p. 268, E. L. Eliel, Ed., Univ. of Notre Dame Press, Notre Dame, IN 1972. The invention additionally encompasses compounds as individual isomers substantially free of other isomers, and alternatively, as mixtures of various isomers. Unless specified to the contrary, the use of wedge and dash lines is used to designate relative stereochemical orientations, not absolute configurations.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆alkyl.

The term “alkyl” refers to a radical of a straight-chain or branched hydrocarbon group having a specified range of carbon atoms (e.g., a “C₁₋₁₆ alkyl” can have from 1 to 16 carbon atoms). In some embodiments, an alkyl group has 1 to 9 carbon atoms (“C₁₋₉ alkyl”). An alkyl group can be saturated or unsaturated, i.e., an alkenyl or alkynyl group as defined herein. Unless specified to the contrary, an “alkyl” group includes both saturated alkyl groups and unsaturated alkyl groups.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. By way of example, a heteroC₁₋₆alkyl (which may also be designated a C₁₋₆heteroalkyl) group includes, but is not limited to, the following structures:

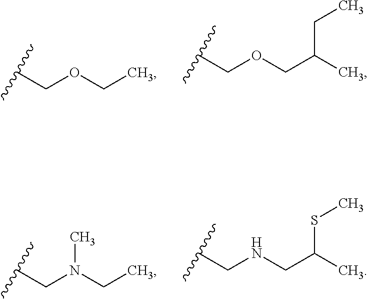

The term “heteroalkyl” preceded by a separate heteroatom refers to a heteroalkyl group bonded through the specified heteroatom. By way of example, a OC₁₋₆heteroalkyl group includes, but it not limited to, the following structures:

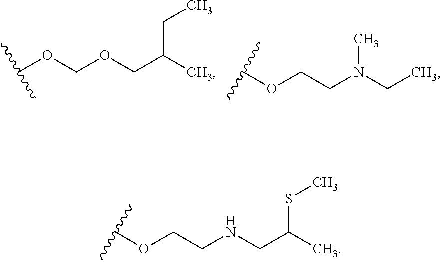

As used herein, the term “heterocyclyl” refers to an aromatic (also referred to as a heteroaryl), unsaturated, or saturated cyclic hydrocarbon that includes at least one heteroatom in the cycle. For example, the term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”).

The term “aryl” refers to a radical of a monocyclic or polycyclic (e.g., bicyclic or tricyclic) 4n+2 aromatic ring system (e.g., having 6, 10, or 14 n electrons shared in a cyclic array) having 6-14 ring carbon atoms and zero heteroatoms provided in the aromatic ring system (“C₆₋₁₄aryl”).

The term “heteroaryl” refers to a radical of a 5-14 membered monocyclic or polycyclic (e.g., bicyclic, tricyclic) 4n+2 aromatic ring system (e.g., having 6, 10, or 14 n electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”).

In general, the inclusion of the prefix “alk” in front of a substituent name indicates there is an alkyl group (as defined herein) connecting the named substituent with the rest of the compound. For example, “alkaryl” (which is a subset of alkyl) refers to an alkyl group substituted by an aryl group, wherein the point of attachment is on the alkyl moiety and “alkheteroaryl” (which is a subset of “alkyl”) refers to an alkyl group substituted by a heteroaryl group, wherein the point of attachment is on the alkyl moiety. The number of carbons atoms may be specified in the alkyl chain, the named substituent, or both. For example, C₁₋₂alkC₆aryl refers to a phenyl ring (which may be substituted) connected via a 1-2 carbon alkylene group.

Affixing the suffix “-ene” to a group indicates the group is a polyvalent moiety, e.g., boned to two or more groups. Alkylene is the polyvalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. “Optionally substituted” refers to a group which may be substituted or unsubstituted. In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

The term “halo” or “halogen” refers to fluorine (fluoro, —F), chlorine (chloro, —Cl), bromine (bromo, —Br), or iodine (iodo, —I).

The term “oxo” refers to the group ═O, and the term “thiooxo” refers to the group ═S.

As used herein, the designation of a polyvalent moiety without specifying the specific order of attachment is intended to cover all possible arrangements. By way of example, a compound represented by the formula:

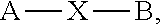

wherein X is NHC(═O) embraces both:

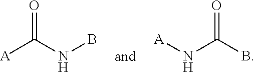

As used herein, a chemical bond depicted, [symbol] represents either a single, double, or triple bond, valency permitting. By way of example,

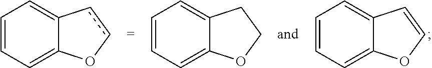

An electron-withdrawing group is a functional group or atom that pulls electron density towards itself, away from other portions of the molecule, e.g., through resonance and/or inductive effects. Exemplary electron-withdrawing groups include F, Cl, Br, I, NO₂, CN, SO₂R, SO₃R, SO₂NR₂, C(O)R^{1a}, C(O)OR, and C(O)NR₂ (wherein R is H or an alkyl, aryl, heteroaryl, cycloalkyl, heterocyclyl group) as well as alkyl group substituted with one or more of those group.

An electron-donating group is a functional group or atom that pushes electron density away from itself, towards other portions of the molecule, e.g., through resonance and/or inductive effects. Exemplary electron-donating groups include unsubstituted alkyl or aryl groups, OR and N(R)₂ and alkyl groups substituted with one or more OR and N(R)₂ groups.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

Some compounds disclosed herein may exist as one or more tautomers. Tautomers are interconvertible structural isomers that differ in the position of one or more protons or other labile atom. By way of example:

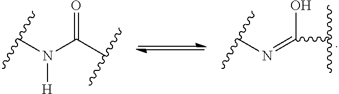

The prevalence of one tautomeric form over another will depend on the specific chemical compound as well as its local chemical environment. Unless specified to the contrary, the depiction of one tautomeric form is inclusive of all possible tautomeric forms.

Unless stated to the contrary, a substituent drawn without explicitly specifying the point of attachment indicates that the substituent may be attached at any possible atom. For example, in a benzofuran depicted as:

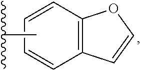

the substituent may be present at any one of the six possible carbon atoms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

Disclosed herein are hydrogel depot system constructed with oxanorbornadiene (OND) linkages, a unique family of small molecule linkers that can rapidly react with thiol groups, triggering the subsequent fragmentation phase automatically. OND linkers can create connections that fragment at predetermined rates dictated by their chemical structures, independently of physiological conditions. Additionally, the fragmentation products are harmless to cells and organisms, making this technology well-suited for biomedical applications. In one implementation, ONDs that connect to dextran sidechains react with thiols at the end of polyethylene glycol (PEG) branches to fabricate hydrogels in physiological conditions, achieving injectability and tunable degradability of the materials due to the nature of ONDs. In some implementations, the cargo includes Qβ virus-like particles (VLPs), a model nanoparticulate subunit vaccine, which are highly stable, strongly immunostimulatory, and capable of displaying multiple antigen epitopes on their surface, making them effective subunit vaccines. Furthermore, their specific size (30 nm diameter) allows them to be large enough to avoid freely diffusing from the hydrogel depot and small enough to diffuse efficiently into draining lymph nodes. Different OND linkers to create hydrogels that degrade in a programmable manner and allow the entrapped VLPs to release in pre-set kinetics. Proper ratios of the combinational linkers provide a variety of vaccine dosing profiles.

Disclosed herein are compositions including a crosslinked network obtained by crosslinking a mixture comprising a multi-valent nucleophile-terminated compound and a first polymer functionalized with oxanorbornene groups of Formula (1):

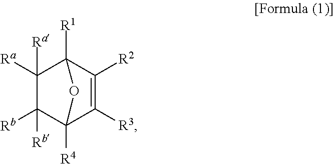

wherein:
R^{a} and R^{b} together form an epoxide or double bond;
R¹ is selected from H, halo, C₁₋₄alkyl, C₁₋₄haloalkyl, C₀₋₄alkC₆₋₁₂aryl, C₀₋₄alkC₃₋₁₂heteroaryl, or -L-polymer;
R⁴ is selected from H, halo, C₁₋₄alkyl, C₁₋₄haloalkyl, C₀₋₄alkC₆₋₁₂aryl, C₀₋₄alkC₃₋₁₂heteroaryl, or -L-polymer;
R^{a′} is selected from H, halo, C₁₋₄alkyl, C₁₋₄haloalkyl, C₀₋₄alkC₆₋₁₂aryl, C₀₋₄alkC₃₋₁₂heteroaryl, or -L-polymer;
R^{b′} is selected from H, halo, C₁₋₄alkyl, C₁₋₄haloalkyl, C₀₋₄alkC₆₋₁₂aryl, C₀₋₄alkC₃₋₁₂heteroaryl, or -L-polymer;
R² is selected from trifluoromethyl, cyano, nitro, C(═O)R^{c}, C(═O)OR^{c}, C(═O)NHR^{c}, C(═O)N(R^{c})₂;
R³ is selected from trifluoromethyl, cyano, nitro, C(═O)R^{d}, C(═O)OR^{d}, C(═O)NHR^{d}, C(═O)N(R^{d})₂;
wherein R^{c} and R^{d} are independently a C₁₋₄alkyl group, C₀₋₄alkC₆₋₁₂aryl, or -L-polymer, or R^{c} and R^{d} together form a ring;
L is a linker;
wherein the oxanorbornene is substituted by a single -L-polymer group.

In some implementations the oxanorbornene of Formula (1) is:

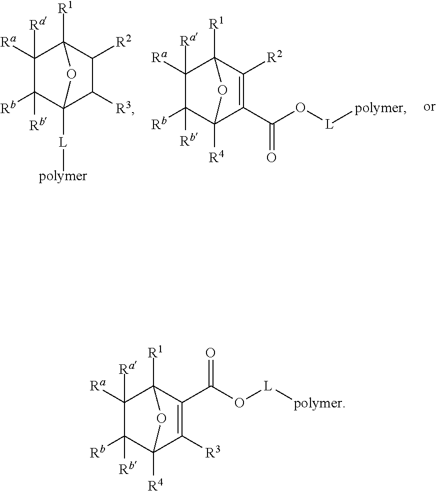

In certain implementations the oxanorbornene of Formula (1) has the Formula (1a) or Formula (1e):

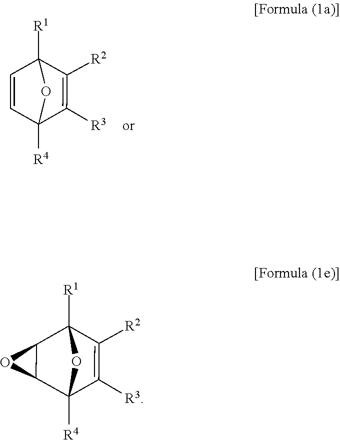

In some implementations the oxanorbornene of Formula (1) is:

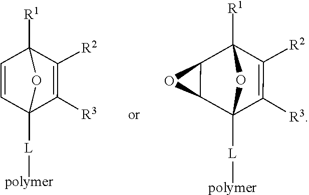

In some implementations of the oxanorbornene of Formula (1), L has the formula —[CH₂]ₓ—X¹—C₀₋₄alk-L*-C₀₋₄alk-X²—, wherein —[CH₂]ₓ— is bonded to the oxanorbornene and x is 0, 1, or 2;
X¹ is null, NH, O, OC(═O), NHC(═O), or NHC(═O)NH;
L* is null, C₁₋₆alkyl, C₁₋₆heteroalkyl, C₆₋₁₂aryl, C₃₋₁₂heteroaryl, C₃₋₁₂cycloalkyl, C₁₋₁₂heterocyclyl, or (CH₂CH₂O)_{z}, wherein z is 1-500, 1-5, 1-10, 5-15, 5-25, 10-50, 10-100, 25-50, 50-100, or 100-500; wherein said L* may be substituted one or more times by halo or hydroxyl; and
X² is null, NH, O, C(═O), OC(═O), NHC(═O), NHC(═O)NH.

In some implementations of the oxanorbornene of Formula (1), L is a (CH₂CH₂O)_{z}, wherein z is 1-500, 1-5, 1-10, 5-15, 5-25, 10-50, 10-100, 25-50, 50-100, or 100-500; or a peptide.

In some implementations of the oxanorbornene of Formula (1) L is —CH₂-L*-C₀₋₄alk-X²— or —CH₂—NHC(═O)—C₀₋₄alk-L*-C₀₋₄alk-X²—, wherein L* is C₁₋₆alkyl or C₃₋₁₂heteroaryl.

In some implementations of the oxanorbornene of Formula (1), L* is the product of a click-cycloaddition.

In some implementations of the oxanorbornene of Formula (1), L* is a 1,2,3 triazole.

In some implementations of the oxanorbornene of Formula (1), L-polymer is:

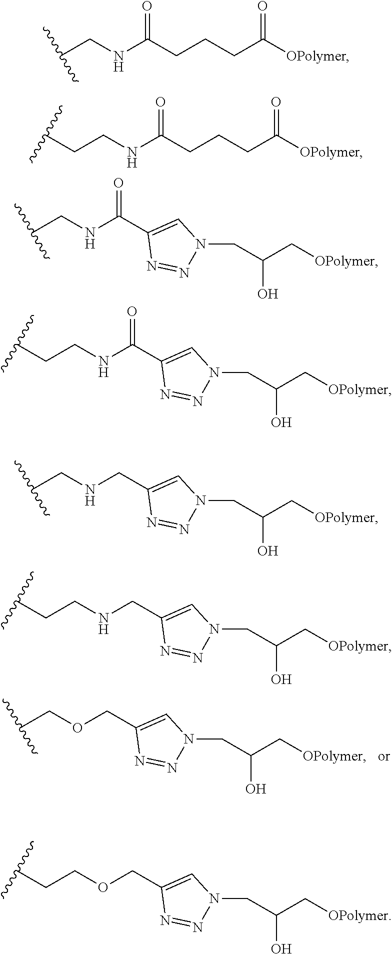

wherein the wavy line indicates the bond to the oxanorbornene, and a is 0, 1, 2, 3, 4, or 5.

In some implementations of the oxanorbornene of Formula (1):
R² is CF₃ and R³ is CO₂Et, CO₂Me, or CO₂CH₂Ph;
R³ is CF₃ and R² is CO₂Et, CO₂Me, or CO₂CH₂Ph.

In some implementations of the oxanorbornene of Formula (1):
R² is CO₂(CH₂)_{b}CO₂Polymer or C(═O)NH(CH₂)_{b}CO₂Polymer, and R³ is CF₃, CO₂Et, CO₂Me, or CO₂CH₂Ph; or
R³ is CO₂(CH₂)_{b}CO₂Polymer or C(═O)NH(CH₂)_{b}CO₂Polymer, and R² is CF₃, CO₂Et, CO₂Me, or CO₂CH₂Ph;
wherein b is 2, 3, 4, 5, 6, or 7.

In some implementations of the oxanorbornene of Formula (1), R¹ is methyl. In some implementations of the oxanorbornene of Formula (1), R¹ is H.

In certain implementations of the oxanorbornene of Formula (1), L has the formula —CH₂— X¹—C₀₋₄alk-L*-C₀₋₄alk-X²—, wherein the methylene group is bonded to the oxanorbornene;
X¹ is null, NH, O, OC(═O), NHC(═O), or NHC(═O)NH;
L* is null, C₁₋₆alkyl, C₁₋₆heteroalkyl, C₆₋₁₂aryl, C₃₋₁₂heteroaryl, C₃₋₁₂cycloalkyl, C₁₋₁₂heterocyclyl, or (CH₂CH₂O)_{z}, wherein z is 1-500, 1-5, 1-10, 5-15, 5-25, 10-50, 10-100, 25-50, 50-100, or 100-500; wherein said L* may be substituted one or more times by halo or hydroxyl; and
X² is null, NH, O, C(═O), OC(═O), NHC(═O), NHC(═O)NH.

In some implementations, the L group in the oxanorbornene of Formula (1) is:

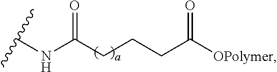

wherein the wavy line indicates the bond to the methylene bonded to the oxanorbornene, and a is 0, 1, 2, 3, 4, or 5. In some implementations the polymer includes hydroxyl functional groups that become part of the ester in the above L group. Exemplary hydroxyl-containing polymers include polysaccharides such as celluloses, dextran, hyaluronic acid, alginate, chitosan, chitin, etc.

In some implementations the L group in the oxanorbornene of Formula (1) is a polyethylene glycol, e.g., (CH₂CH₂O)_{z}, wherein z is 1-500, 1-5, 1-10, 5-15, 5-25, 10-50, 10-100, 25-50, 50-100, or 100-500. In some implementations, the L group in the oxanorbornene of Formula (1) is a peptide.

In some implementations the L* group in the oxanorbornene of Formula (1) is the product of a click-cycloaddition, for example the product of a cycloaddition reaction between an azide, nitrone, 1,2,4,5 tetrazine, and an alkyne (including a cyclooctyne) or trans-cyclooctene. In certain implementations of the oxanorbornene of Formula (1) L* is a triazole.

In some implementations, the L* group in the oxanorbornene of Formula (1) is:

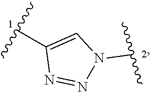 [SMILES: *c1cn(*)nn1]

wherein wavy line 1 indicates the bond to —[CH₂]_{z}—X¹—C₀₋₄alk- and wavy line 2 represents the bond to C₀₋₄alk-X². In other implementations, wavy line 2 indicates the bond to —[CH₂]_{z}—X¹—C₀₋₄alk- and wavy line 1 represents the bond to C₀₋₄alk-X².

In some implementations the R¹ group in the oxanorbornene of Formula (1) R¹ is methyl.

In some implementations of the oxanorbornene of Formula (1) R² is CF₃ and R³ is CO₂Et or CO₂Me. In some implementations of the oxanorbornene of Formula (1) R³ is CF₃ and R² is CO₂Et or CO₂Me. In certain implementations of the oxanorbornene of Formula (1) one of R² or R³ is CO₂(CH₂)_{b}CO₂Polymer or C(═O)NH(CH₂)_{b}CO₂Polymer, wherein b is 2, 3, 4, 5, 6, or 7, and the other is CF₃, CO₂Et, or CO₂Me.

In some implementations the first polymer is a polysaccharide, a polypeptide, a synthetic polymer, or a combination thereof. In some implementations, the first polymer is cellulose, cellulose derivative, hyaluronic acid, dextran, alginate, carrageenan, chitosan, heparin, pectin, starch, gelatin, xanthan gum, or a combination thereof. Exemplary cellulose derivatives include hydroxyethyl cellulose, hydroxypropyl cellulose, and carboxymethylcellulose.

In some implementations, the first polymer includes polyvinyl alcohol, poly(vinylpyrrolidone), poly(lactic-co-glycolic acid), polyethylene oxide, polyethylene glycol, polyacrylates (such as polyacrylic acid, PAA, and poly(methyl methacrylate), PMMA), polyacrylamides, polymethacrylamides, linear and branched poly(ethylene glycol) (PEG), linear and branched polyethylenimine (PEI), polypropylene sulfides, poly(lactic acid) (PLA), poly(lactic-co-glycolic acid) (PLGA), poly(meth)acrylic acid, poly(meth)acrylamide, poly(meth)acrylate, poly(caprolactone)s, poly(ethylene terephthalate), polyurethanes, poly(norbornene)s, poly(2-oxazoline)s, poly(acetal)s, a copolymer thereof, or a combination thereof

In some implementations, the first polymer includes polyvinyl alcohol, poly(vinylpyrrolidone), poly(lactic acid), poly(lactic-co-glycolic acid) (PLGA), poly(meth)acrylic acid, poly(meth)acrylamide, poly(meth)acrylate, a copolymer thereof, or a combination thereof.

In some implementations, the first polymer include a protein or glycoprotein.

The first polymer may be provided in a variety of molecular weights. In some implementations, the first polymer has a Mₙ from 5 kDa to 3,000 kDa, from 5 kDa to 100 kDa, from 5 kDa to 25 kDa, from 10 kDa to 25 kDa, from 20 kDa to 50 kDa, from 50 kDa to 150 kDa, from 100 kDa to 250 kDa, from 250 kDa to 1,000 kDa, from 1,000 kDa to 2,000 kDa, or from 2,000 kDa to 3,000 kDa.

The first polymer may include the oxanorbornene in varying degrees of substitutions. In some implementations, the first polymer includes oxanorbornene groups of Formula (1) in an amount (per monomer unit) from 0.5-100%, 50-100%, 75-100%, 50-75%, 25-75%, 1-50%, from 1-25%, from 1-20%, from 1-10%, from 1-5%, from 0.5-2.5%, from 5-10%, from 5-20%, from 10-15%, from 10-20%, from 10-25%, from 15-25%, or from 25-50%. By way of example, if the first polymer includes oxanorbornene groups of Formula (1) in an amount (per monomer unit) of 100%, every monomer unit of the polymer will be substituted with an oxanorbornene group.

In certain implementations, the multi-valent nucleophile-terminated compound is a multi-arm thiol, for example a multi-arm PEG thiol. In some implementations, the multi-arm PEG thiol is a 2-arm PEG thiol, a 3-arm PEG thiol, a 4-arm PEG thiol, a 6-arm PEG thiol, an 8-arm PEG thiol. In some implementations combinations of different multi-valent nucleophile-terminated compounds and combinations of different multi-arm thiols may be used.

In certain implementations, the multi-valent nucleophile terminated compound has the formula:

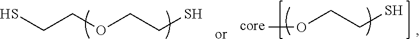

wherein the core has the formula:

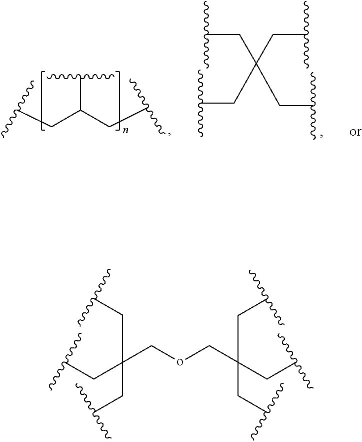

n is an integer from 1-6; and
the multi-valent nucleophile terminated compound has an average MW from 200-50,000 Da, from 200-1,000 Da, from 200-500 Da, from 500-1,000 Da, from 500-1,500 Da, from 1,000-1,500 Da, from 200-2,500 Da, from 500-2,500 Da, from 1,000-2,000 Da, from 1,000-2,500 Da, from 1,500-2,500 Da, from 1,000-5,000 Da, from 2,500-10,000 Da, from 5,000-10,000 Da, from 10,000-25,000 Da, from 10,000-50,000 Da, or from 25,000-50,000 Da.

The crosslinked network may be obtained by combining the first polymer including oxanorbornene groups of Formula (1) and the multi-valent nucleophile-terminated compound in a weight ratio from 10:1 to 1:10, from 10:1 to 1:1, from 10:1 to 5:1, from 5:1 to 1:1, from 2.5:1 to 1:1, from 1.5:1 to 1:1, from 1.25:1 to 1:1.25, from 1:1 to 1:10, from 1:5 to 1:10, from 1:1 to 1:5, from 1:1 to 1:2.5, or from 1:1 to 1:1.5.

In some implementations, the mixture used to prepare the crosslinked network may further include a second polymer functionalized with oxanorbornene groups of Formula (2):

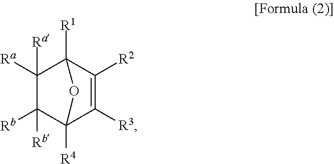

wherein:
R^{a} and R^{b} together form an epoxide or double bond;
R¹ is selected from H, halo, C₁₋₄alkyl, C₁₋₄haloalkyl, C₀₋₄alkC₆₋₁₂aryl, C₀₋₄alkC₃₋₁₂heteroaryl, or -L-polymer;
R⁴ is selected from H, halo, C₁₋₄alkyl, C₁₋₄haloalkyl, C₀₋₄alkC₆₋₁₂aryl, C₀₋₄alkC₃₋₁₂heteroaryl, or -L-polymer;
R^{a′} is selected from H, halo, C₁₋₄alkyl, C₁₋₄haloalkyl, C₀₋₄alkC₆₋₁₂aryl, C₀₋₄alkC₃₋₁₂heteroaryl, or -L-polymer;
R^{b′} is selected from H, halo, C₁₋₄alkyl, C₁₋₄haloalkyl, C₀₋₄alkC₆₋₁₂aryl, C₀₋₄alkC₃₋₁₂heteroaryl, or -L-polymer;
R² is selected from trifluoromethyl, cyano, nitro, C(═O)R^{c}, C(═O)OR^{c}, C(═O)NHR^{c}, C(═O)N(R^{c})₂;
R³ is selected from trifluoromethyl, cyano, nitro, C(═O)R^{d}, C(═O)OR^{d}, C(═O)NHR^{d}, C(═O)N(R^{d})₂;
wherein R^{c} and R^{d} are independently a C₁₋₄alkyl group, C₀₋₄alkC₆₋₁₂aryl, or -L-polymer, or R^{c} and R^{d} together form a ring;
L is a linker;
wherein the oxanorbornene is substituted by a single -L-polymer group; and
wherein the first polymer functionalized with oxanorbornene groups is not the same as the second polymer functionalized with oxanorbornene groups.

The first polymer and second polymer are different. In some implementations, the identities of the various R groups in each oxanorbornene will not be the same but the polymer will be the same (e.g., both polymers will be a dextran). In some implementations, the polymer will not be the same, either being composed of different monomeric units (e.g., dextran and hyaluronic acid) or having distinct molecular weights (e.g., two different dextrans). In the latter implementation, a polymodal distribution of polymers having the same oxanorbornene group will be present in the mixture. In some implementations, both the oxanorbornenes and the polymers will be different.

In some implementations the oxanorbornene of Formula (2) is:

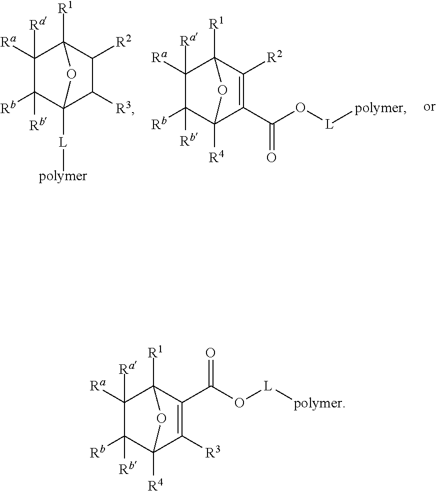

In some implementations the oxanorbornene of Formula (2) has the Formula (2a) or Formula (2e):

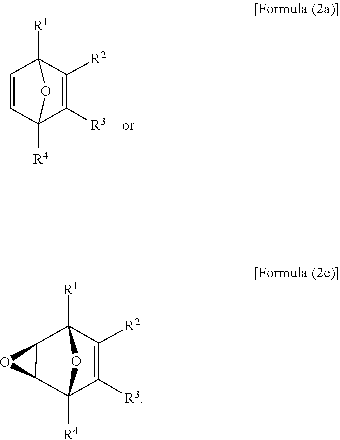

In some implementations the oxanorbornene of Formula (2) is:

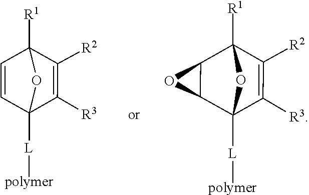

In some implementations of the oxanorbornene of Formula (2), L has the formula —[CH₂]ₓ—X¹—C₀₋₄alk-L*-C₀₋₄alk-X²—, wherein —[CH₂]ₓ— is bonded to the oxanorbornene and x is 0, 1, or 2;
X¹ is null, NH, O, OC(═O), NHC(═O), or NHC(═O)NH;
L* is null, C₁₋₆alkyl, C₁₋₆heteroalkyl, C₆₋₁₂aryl, C₃₋₁₂heteroaryl, C₃₋₁₂cycloalkyl, C₁₋₁₂heterocyclyl, or (CH₂CH₂O)_{z}, wherein z is 1-500, 1-5, 1-10, 5-15, 5-25, 10-50, 10-100, 25-50, 50-100, or 100-500; wherein said L* may be substituted one or more times by halo or hydroxyl; and
X² is null, NH, O, C(═O), OC(═O), NHC(═O), NHC(═O)NH.

In some implementations of the oxanorbornene of Formula (2), L is a (CH₂CH₂O)_{z}, wherein z is 1-500, 1-5, 1-10, 5-15, 5-25, 10-50, 10-100, 25-50, 50-100, or 100-500; or a peptide

In some implementations of the oxanorbornene of Formula (2) L is —CH₂-L*-C₀₋₄alk-X²— or —CH₂—NHC(═O)—C₀₋₄alk-L*-C₀₋₄alk-X²—, wherein L* is C₁₋₆alkyl or C₃₋₁₂heteroaryl.

In some implementations of the oxanorbornene of Formula (2), L* is the product of a click-cycloaddition.

In some implementations of the oxanorbornene of Formula (2), L* is a 1,2,3 triazole.

In some implementations of the oxanorbornene of Formula (2), L-polymer is:

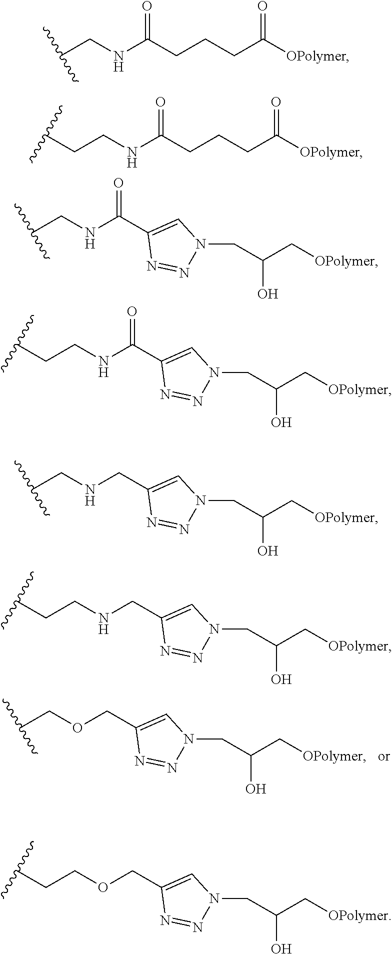

wherein the wavy line indicates the bond to the oxanorbornene, and a is 0, 1, 2, 3, 4, or 5.

In some implementations of the oxanorbornene of Formula (2):
R² is CF₃ and R³ is CO₂Et, CO₂Me, or CO₂CH₂Ph; and
R³ is CF₃ and R² is CO₂Et, CO₂Me, or CO₂CH₂Ph.

In some implementations of the oxanorbornene of Formula (2):
R² is CO₂(CH₂)_{b}CO₂Polymer or C(═O)NH(CH₂)_{b}CO₂Polymer, and R³ is CF₃, CO₂Et, CO₂Me, or CO₂CH₂Ph; or
R³ is CO₂(CH₂)_{b}CO₂Polymer or C(═O)NH(CH₂)_{b}CO₂Polymer, and R² is CF₃, CO₂Et, CO₂Me, or CO₂CH₂Ph;
wherein b is 2, 3, 4, 5, 6, or 7.

In some implementations of the oxanorbornene of Formula (2) R¹ is methyl. In some implementations of the oxanorbornene of Formula (2) R¹ is H.

In some implementations, the L group in the oxanorbornene of Formula (2) is:

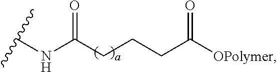

wherein the wavy line indicates the bond to the methylene bonded to the oxanorbornene, and a is 0, 1, 2, 3, 4, or 5. In some implementations the polymer includes hydroxyl functional groups that become part of the ester in the above L group. Exemplary hydroxyl-containing polymers include polysaccharides such as celluloses, dextran, hyaluronic acid, alginate, chitosan, chitin, etc.

In some implementations the L group in the oxanorbornene of Formula (2) is a polyethylene glycol, e.g., (CH₂CH₂O)_{z}, wherein z is 1-500, 1-5, 1-10, 5-15, 5-25, 10-50, 10-100, 25-50, 50-100, or 100-500. In some implementations, the L group in the oxanorbornene of Formula (2) is a peptide.

In some implementations the L* group in the oxanorbornene of Formula (2) is the product of a click-cycloaddition, for example the product of a cycloaddition reaction between an azide, nitrone, 1,2,4,5 tetrazine, and an alkyne (including a cyclooctyne) or trans-cyclooctene. In certain implementations the L group in the oxanorbornene of Formula (2) the L* is a triazole.

In some implementations, the L* group in the oxanorbornene of Formula (2) is:

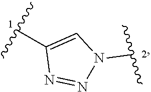 [SMILES: *c1cn(*)nn1]

wherein wavy line 1 indicates the bond to —[CH₂]_{z}—X¹—C₀₋₄alk- and wavy line 2 represents the bond to C₀₋₄alk-X². In other implementations, wavy line 2 indicates the bond to —[CH₂]_{z}—X¹—C₀₋₄alk- and wavy line 1 represents the bond to C₀₋₄alk-X².

In some implementations of the oxanorbornene of Formula (2) R¹ is methyl.

In some implementations of the oxanorbornene of Formula (2) R² is CF₃ and R³ is CO₂Et or CO₂Me. In some implementations of the oxanorbornene of Formula (2) R³ is CF₃ and R² is CO₂Et or CO₂Me. In certain implementations of the oxanorbornene of Formula (2) one of R² or R³ is CO₂(CH₂)_{b}CO₂Polymer or C(═O)NH(CH₂)_{b}CO₂Polymer, wherein b is 2, 3, 4, 5, 6, or 7, and the other of R² or R³ is CF₃, CO₂Et, or CO₂Me.

In some implementations the second polymer is a polysaccharide, a polypeptide, a synthetic polymer, or a combination thereof. In some implementations, the second polymer is cellulose, cellulose derivative, hyaluronic acid, dextran, alginate, carrageenan, chitosan, heparin, pectin, starch, gelatin, xanthan gum, or a combination thereof. Exemplary cellulose derivatives include hydroxyethyl cellulose, hydroxypropyl cellulose, and carboxymethylcellulose.

In some implementations, the second polymer includes polyvinyl alcohol, poly(vinylpyrrolidone), poly(lactic-co-glycolic acid), polyethylene oxide, polyethylene glycol, polyacrylates (such as polyacrylic acid, PAA, and poly(methyl methacrylate), PMMA), polyacrylamides, polymethacrylamides, linear and branched poly(ethylene glycol) (PEG), linear and branched polyethylenimine (PEI), polypropylene sulfides, poly(lactic acid) (PLA), poly(lactic-co-glycolic acid) (PLGA), poly(meth)acrylic acid, poly(meth)acrylamide, poly(meth)acrylate, poly(caprolactone)s, poly(ethylene terephthalate), polyurethanes, poly(norbornene)s, poly(2-oxazoline)s, poly(acetal)s, a copolymer thereof, or a combination thereof

In some implementations, the second polymer includes polyvinyl alcohol, poly(vinylpyrrolidone), poly(lactic acid), poly(lactic-co-glycolic acid) (PLGA), poly(meth)acrylic acid, poly(meth)acrylamide, poly(meth)acrylate, a copolymer thereof, or a combination thereof.

In some implementations, the second polymer includes a protein or glycoprotein.

The second polymer may be provided in a variety of molecular weights. In some implementations, the first polymer has a Mₐ from 5 kDa to 3,000 kDa, from 5 kDa to 100 kDa, from 5 kDa to 25 kDa, from 10 kDa to 25 kDa, from 20 kDa to 50 kDa, from 50 kDa to 150 kDa, from 100 kDa to 250 kDa, from 250 kDa to 1,000 kDa, from 1,000 kDa to 2,000 kDa, or from 2,000 kDa to 3,000 kDa.

The second polymer may include the oxanorbornene of Formula (2) in varying degrees of substitutions. In some implementations, the first polymer includes oxanorbornene groups of Formula (2) in an amount (per monomer unit) from 0.5-100%, 50-100%, 75-100%, 50-75%, 25-75%, 1-50%, from 1-25%, from 1-20%, from 1-10%, from 1-5%, from 0.5-2.5%, from 5-10%, from 5-20%, from 10-15%, from 10-20%, from 10-25%, from 15-25%, or from 25-50%.

In certain implementations, the composition can include one or more cargo moieties. Unless specified to the contrary, cargo is inclusive of both single and combinations of molecular entities. In certain implementations, cargo is non-covalently entrapped in the crosslinked network. In other implementations, the cargo is covalently bonded to the network. In further implementations, a cargo is non-covalently entrapped in the crosslinked network, and a cargo (which may be the same or different than the entrapped cargo) is covalently bonded to the network. The crosslinked network compositions disclosed herein can be programmed to degrade at certain rates, thereby releasing the cargo in a controlled fashion. The period over which the composition releases the cargo is the period over which the composition delivers the clinically desired amount of the cargo to the subject. At the end of the release period, the composition is no longer delivering clinically desired amount of the cargo. Clinically desired amounts may be determined via systemic blood concentrations and/or other quantitative assessments.

In certain implementations the cargo is continuously released over a period of 2 hours, 4 hours, 6 hours, 8 hours, 10 hours, 12 hours, 16 hours or 24 hours.

In certain implementations the cargo is continuously released over a period of 1 day, 2 days, 3 days, 4 days, 5 days, 6 days, or 7 days.

In certain implementations the cargo is continuously released over a period of 1 week, 2 weeks, 4 weeks, 8 weeks, 12 weeks, or 16 weeks.

In certain implementations the cargo is continuously released over a period of 1 month, 2 months, 3 months, 4 months, 5 months, 6 months, 7 months, 8 months, 9 months, 10 months, 11 months, or 12 months.

In some implementations including a cargo covalently bonded to the network, the crosslinked network may be formed from a mixture including a third polymer functionalized with oxanorbornene groups of Formula (3):

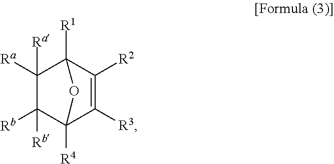

wherein:
R^{a} and R^{b} together form an epoxide or double bond;
R¹ is selected from H, halo, C₁₋₄alkyl, C₁₋₄haloalkyl, C₀₋₄alkC₆₋₁₂aryl, C₀₋₄alkC₃₋₁₂heteroaryl, -L-polymer, or L¹-cargo;
R⁴ is selected from H, halo, C₁₋₄alkyl, C₁₋₄haloalkyl, C₀₋₄alkC₆₋₁₂aryl, C₀₋₄alkC₃₋₁₂heteroaryl, -L-polymer, or L¹-cargo;
R^{a′} is selected from H, halo, C₁₋₄alkyl, C₁₋₄haloalkyl, C₀₋₄alkC₆₋₁₂aryl, C₀₋₄alkC₃₋₁₂heteroaryl, or -L-polymer, or L¹-cargo;
R^{b′} is selected from H, halo, C₁₋₄alkyl, C₁₋₄haloalkyl, C₀₋₄alkC₆₋₁₂aryl, C₀₋₄alkC₃₋₁₂heteroaryl, or -L-polymer, or L¹-cargo;
R² is selected from trifluoromethyl, cyano, nitro, C(═O)R^{c}, C(═O)OR^{c}, C(═O)NHR^{c}, C(═O)N(R^{c})₂;
R³ is selected from trifluoromethyl, cyano, nitro, C(═O)R^{d}, C(═O)OR^{d}, C(═O)NHR^{d}, C(═O)N(R^{d})₂;
wherein R^{c} and R^{d} are independently a C₁₋₄alkyl group, C₀₋₄alkC₆₋₁₂aryl, -L-polymer, or L¹-cargo, or R^{c} and R^{d} together form a ring;
L is a linker;
L¹ is a linker; and
wherein the oxanorbornene is substituted by a single -L-polymer group.

In some implementations, the mixture includes the first polymer and the third polymer. In some implementations, the mixture includes the first polymer, the second polymer and the third polymer. In some implementations, the third polymer will be the same as either (or both) of the first and second polymers. For example, the first polymer can be a dextran functionalized with oxanorbornenes of Formula (1) and the third polymer can be a dextran functionalized with oxanorbornenes of Formula (3). In other implementations, the third polymer is not the same as either.

In certain implementations of the oxanorbornene of Formula (3) is:

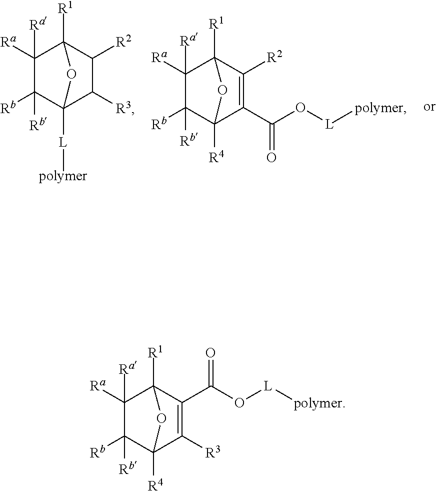

In certain implementations of the oxanorbornene of Formula (3) has the formula (3a) or Formula (3e):

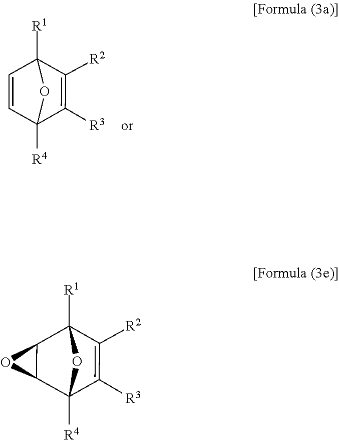

In certain implementations of the oxanorbornene of Formula (3) has the formula:

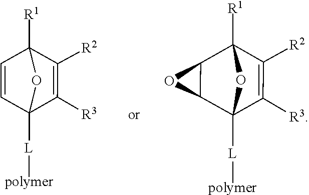

In certain implementations of the oxanorbornene of Formula (3) L has the formula —[CH₂]ₓ—X¹—C₀₋₄alk-L*-C₀₋₄alk-X²—, wherein —[CH₂]ₓ— is bonded to the oxanorbornene and x is 0, 1, or 2;
X¹ is null, NH, O, OC(═O), NHC(═O), or NHC(═O)NH;
L* is null, C₁₋₆alkyl, C₁₋₆heteroalkyl, C₆₋₁₂aryl, C₃₋₁₂heteroaryl, C₃₋₁₂cycloalkyl, C₁₋₁₂heterocyclyl, or (CH₂CH₂O)_{z}, wherein z is 1-500, 1-5, 1-10, 5-15, 5-25, 10-50, 10-100, 25-50, 50-100, or 100-500; wherein said L* may be substituted one or more times by halo or hydroxyl; and
X² is null, NH, O, C(═O), OC(═O), NHC(═O), NHC(═O)NH

In certain implementations of the oxanorbornene of Formula (3) L is a (CH₂CH₂O)_{z}, wherein z is 1-500, 1-5, 1-10, 5-15, 5-25, 10-50, 10-100, 25-50, 50-100, or 100-500; or a peptide.

In certain implementations of the oxanorbornene of Formula (3) L is —CH₂-L*-C₀₋₄alk-X²— or —CH₂—NHC(═O)—C₀₋₄alk-L*-C₀₋₄alk-X²—, wherein L* is C₁₋₆alkyl or C₃₋₁₂heteroaryl.

In certain implementations of the oxanorbornene of Formula (3) L* is the product of a click-cycloaddition.

In certain implementations of the oxanorbornene of Formula (3) L* is a 1,2,3 triazole.

In certain implementations of the oxanorbornene of Formula (3) L-polymer is:

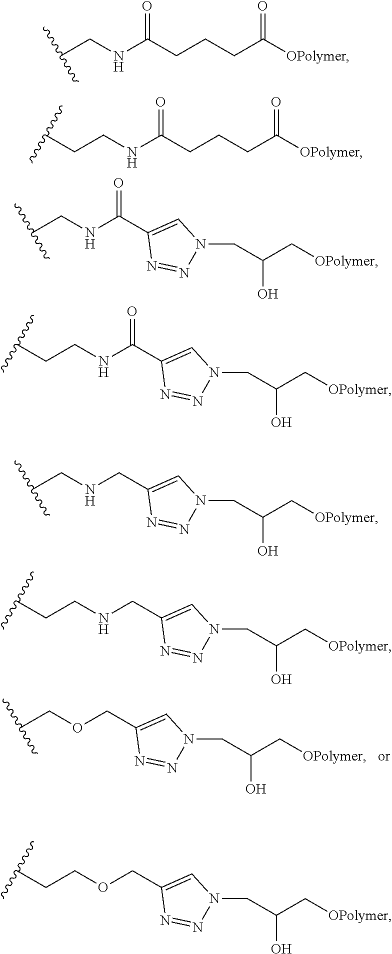

wherein the wavy line indicates the bond to the oxanorbornene, and a is 0, 1, 2, 3, 4, or 5.

In certain implementations of the oxanorbornene of Formula (3):
R² is CF₃ and R³ is CO₂Et, CO₂Me, or CO₂CH₂Ph; and
R³ is CF₃ and R² is CO₂Et, CO₂Me, or CO₂CH₂Ph

In certain implementations of the oxanorbornene of Formula (3):
R² is CO₂(CH₂)_{b}CO₂Polymer or C(═O)NH(CH₂)_{b}CO₂Polymer, and R³ is CF₃, CO₂Et, CO₂Me, or CO₂CH₂Ph; or
R³ is CO₂(CH₂)_{b}CO₂Polymer or C(═O)NH(CH₂)_{b}CO₂Polymer, and R² is CF₃, CO₂Et, CO₂Me, or CO₂CH₂Ph;
wherein b is 2, 3, 4, 5, 6, or 7.

In certain implementations of the oxanorbornene of Formula (3) R¹ is methyl. In some implementations of the oxanorbornene of Formula (3) R¹ is H.

In certain implementations of the oxanorbornene of Formula (3), L¹-cargo has the formula —[CH₂]_{y}—C₀₋₄alk-X¹-L*-C₀₋₄alk-X²-cargo, wherein y is 0, 1, or 2, and —[CH₂]_{y} is bonded to the oxanorbornene;
X¹ is null, NH, O, OC(═O), NHC(═O), or NHC(═O)NH;
L* is null, C₁₋₆alkyl, C₁₋₆heteroalkyl, C₆₋₁₂aryl, C₃₋₁₂heteroaryl, C₃₋₁₂cycloalkyl, C₁₋₁₂heterocyclyl, or (CH₂CH₂O)_{z}, wherein z is 1-500, 1-5, 1-10, 5-15, 5-25, 10-50, 10-100, 25-50, 50-100, or 100-500; wherein said L* may be substituted one or more times by halo or hydroxyl; and
X² is null, NH, O, C(═O), OC(═O), NHC(═O), NHC(═O)NH.

In certain implementations of the oxanorbornene of Formula (3), L has the formula —CH₂— X¹—C₀₋₄alk-L*-C₀₋₄alk-X²—, wherein the methylene group is bonded to the oxanorbornene;
X¹ is null, NH, O, OC(═O), NHC(═O), or NHC(═O)NH;
L* is null, C₁₋₆alkyl, C₁₋₆heteroalkyl, C₆₋₁₂aryl, C₃₋₁₂heteroaryl, C₃₋₁₂cycloalkyl, C₁₋₁₂heterocyclyl, or (CH₂CH₂O)_{z}, wherein z is 1-500, 1-5, 1-10, 5-15, 5-25, 10-50, 10-100, 25-50, 50-100, or 100-500; wherein said L* may be substituted one or more times by halo or hydroxyl; and
X² is null, NH, O, C(═O), OC(═O), NHC(═O), NHC(═O)NH.

In some implementations, the L group in the oxanorbornene of Formula (3) is:

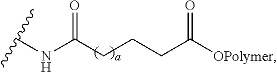

wherein the wavy line indicates the bond to the methylene bonded to the oxanorbornene, and a is 0, 1, 2, 3, 4, or 5. In some implementations the polymer includes hydroxyl functional groups that become part of the ester in the above L group. Exemplary hydroxyl-containing polymers include polysaccharides such as celluloses, dextran, hyaluronic acid, alginate, chitosan, chitin, etc.

In some implementations the L group in the oxanorbornene of Formula (3) is a polyethylene glycol, e.g., (CH₂CH₂O)_{z}, wherein z is 1-500, 1-5, 1-10, 5-15, 5-25, 10-50, 10-100, 25-50, 50-100, or 100-500. In some implementations, the L group in the oxanorbornene of Formula (3) is a peptide.

In some implementations the L* group in the oxanorbornene of Formula (3) is the product of a click-cycloaddition, for example the product of a cycloaddition reaction between an azide, nitrone, 1,2,4,5 tetrazine, and an alkyne (including a cyclooctyne) or trans-cyclooctene. In certain implementations the L group in the oxanorbornene of Formula (3) the L* is a triazole.

In some implementations, the L* group in the oxanorbornene of Formula (3) is:

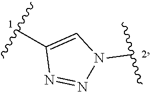 [SMILES: *c1cn(*)nn1]

wherein wavy line 1 indicates the bond to —[CH₂]_{z}—X¹—C₀₋₄alk- and wavy line 2 represents the bond to C₀₋₄alk-X². In other implementations, wavy line 2 indicates the bond to —[CH₂]_{z}—X¹—C₀₋₄alk- and wavy line 1 represents the bond to C₀₋₄alk-X².

In some implementations, the L group in the oxanorbornene of Formula (3) is:

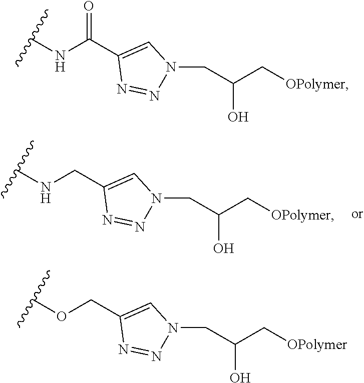

wherein the wavy line indicates the bond to the methylene bonded to the oxanorbornene,

In some implementations the R¹ group in the oxanorbornene of Formula (3) R¹ is methyl.

In some implementations of the oxanorbornene of Formula (3) R² is CF₃ and R³ is CO₂Et or CO₂Me. In some implementations of the oxanorbornene of Formula (2) R³ is CF₃ and R² is CO₂Et or CO₂Me. In certain implementations of the oxanorbornene of Formula (3) one of R² or R³ is CO₂(CH₂)_{b}CO₂Polymer or C(═O)NH(CH₂)_{b}CO₂Polymer, wherein b is 2, 3, 4, 5, 6, or 7, and the other is CF₃, CO₂Et, or CO₂Me. In certain implementations of the oxanorbornene of Formula (3) one of R² or R³ is CO₂(CH₂)_{b}CO₂Cargo or C(═O)NH(CH₂)_{b}CO₂Cargo, wherein b is 2, 3, 4, 5, 6, or 7, and the other of R² or R³ is CF₃, CO₂Et, or CO₂Me.

In some implementations the third polymer is a polysaccharide, a polypeptide, a synthetic polymer, or a combination thereof. In some implementations, the third polymer is cellulose, cellulose derivative, hyaluronic acid, dextran, alginate, carrageenan, chitosan, heparin, pectin, starch, gelatin, xanthan gum, or a combination thereof. Exemplary cellulose derivatives include hydroxyethyl cellulose, hydroxypropyl cellulose, and carboxymethylcellulose.

In some implementations, the third polymer includes polyvinyl alcohol, poly(vinylpyrrolidone), poly(lactic-co-glycolic acid), polyethylene oxide, polyethylene glycol, polyacrylates (such as polyacrylic acid, PAA, and poly(methyl methacrylate), PMMA), polyacrylamides, polymethacrylamides, linear and branched poly(ethylene glycol) (PEG), linear and branched polyethylenimine (PEI), polypropylene sulfides, poly(lactic acid) (PLA), poly(lactic-co-glycolic acid) (PLGA), poly(meth)acrylic acid, poly(meth)acrylamide, poly(meth)acrylate, poly(caprolactone)s, poly(ethylene terephthalate), polyurethanes, poly(norbornene)s, poly(2-oxazoline)s, poly(acetal)s, a copolymer thereof, or a combination thereof

In some implementations, the third polymer includes polyvinyl alcohol, poly(vinylpyrrolidone), poly(lactic acid), poly(lactic-co-glycolic acid) (PLGA), poly(meth)acrylic acid, poly(meth)acrylamide, poly(meth)acrylate, a copolymer thereof, or a combination thereof.

In some implementations, the third polymer includes a protein or glycoprotein.

The third polymer may be provided in a variety of molecular weights. In some implementations, the third polymer has a Mₐ from 5 kDa to 3,000 kDa, from 5 kDa to 100 kDa, from 5 kDa to 25 kDa, from 10 kDa to 25 kDa, from 20 kDa to 50 kDa, from 50 kDa to 150 kDa, from 100 kDa to 250 kDa, from 250 kDa to 1,000 kDa, from 1,000 kDa to 2,000 kDa, or from 2,000 kDa to 3,000 kDa.

The third polymer may include the oxanorbornene of Formula (3) in varying degrees of substitutions. In some implementations, the first polymer includes oxanorbornene groups of Formula (1) in an amount (per monomer unit) from 0.01-100%, from 0.01-0.1%, from 0.01-1%, from 0.1-1%, from 0.5-1%, from 0.5-100%, 50-100%, 75-100%, 50-75%, 25-75%, 1-50%, from 1-25%, from 1-20%, from 1-10%, from 1-5%, from 0.5-2.5%, from 5-10%, from 5-20%, from 10-15%, from 10-20%, from 10-25%, from 15-25%, or from 25-50%.

In certain implementations, the cargo includes at least one therapeutic agent, antigenic agent, diagnostic agent, or analytical agent. In some implementations, the cargo includes a protein, nucleic acid, polynucleic acid, polysaccharide, glycoprotein, proteoglycan, or lipid.

In some implementations, the composition may further include nanoparticles. In some implementations, the composition includes lipid nanoparticles, polymer nanoparticles, protein nanoparticles, or a combination thereof.

Also disclosed herein are methods of delivering therapeutic agents to subjects using the compositions disclosed herein. The compositions may be administered to the subject by a variety of routes, including topically, subcutaneously, intramuscularly, intradermally, intravenously, or by surgical implantation. In certain implementations, the therapeutic agent is biologic therapeutic agent (for example a therapeutic protein, monoclonal antibody, or antibody-drug-conjugate).

Also disclosed herein are methods of vaccinating a subject by administering to the subject a composition disclosed herein that includes an antigen, an adjuvant, or a combination thereof.

Also disclosed herein are methods of making the compositions disclosed herein by forming a mixture including water, the first polymer functionalized with an oxanorbornene of Formula (1) and a multi-valent nucleophile-terminated compound to form a crosslinked network. The mixture may further include the second polymer functionalized with oxanorbornene groups of Formula (2), the third polymer functionalized with an oxanorbornene of Formula (3), one or more separate cargo species, or a combination thereof.

In certain implementations, the mixture may be formed using a syringe-to-syringe process. By way of example, a first syringe containing a solution of the first polymer may be joined via Luer lock or similar connector to a second syringe containing a solution of the multi-valent nucleophile-terminated compound. The solutions may be passed between the syringes to ensure complete mixing.

In certain implementations, the cargo can include one or more therapeutic agents, for example small molecule agents such as antibiotics, anticancer drugs, contraceptives, and the like. The compositions can also include biologic therapeutic agents such as proteins, nucleic acids, and polysaccharides.

In certain implementations, the cargo can include Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

In certain implementations, the cargo can include one or more hormones, for example human growth hormone.

In certain implementations, the cargo can include one or more biologically active polypeptides. In certain embodiments, the compositions can include one or more of bone morphogenetic protein 1 (BMP1), bone morphogenetic protein 2 (BMP2), bone morphogenetic protein 3 (BMP3), bone morphogenetic protein 4 (BMP4), bone morphogenetic protein 5 (BMP5), bone morphogenetic protein 6 (BMP6), bone morphogenetic protein 7 (BMP7), bone morphogenetic protein 8a (BMP8a), epidermal growth factor (EGF), platelet-derived growth factor alpha polypeptide (PDGFA), platelet-derived growth factor beta polypeptide (PDGFB), platelet derived growth factor C (PDGFC), platelet derived growth factor D (PDGFD), platelet derived growth factor AB (PDGFAB), vascular endothelial growth factor A (VEGF-A), placenta growth factor (PIGF), vascular endothelial growth factor B (VEGF-B), vascular endothelial growth factor C (VEGF-C), vascular endothelial growth factor D (VEGF-D), transforming growth factor beta 1 (TGF-31), transforming growth factor beta 2 (TGF-@2), transforming growth factor beta 3 (TGF-@3), anti-mullerian hormone (AMH), artemin (ARTN), growth-differentiation factor-1 (GDF1), growth-differentiation factor-2 (GDF2), growth-differentiation factor-3 (GDF3), growth-differentiation factor-3A (GDF3A), growth-differentiation factor-5 (GDF5), growth-differentiation factor-6 (GDF6), growth-differentiation factor-7 (GDF7), growth-differentiation factor-8 (GDF8), growth-differentiation factor-9 (GDF9), growth-differentiation factor-10 (GDF10), growth-differentiation factor-11 (GDF11), growth-differentiation factor-15 (GDF15), neurotrophic factor (GDFN), inhibin alpha chain (INHA), inhibin beta A chain (INHBA), inhibin beta B chain (INHBB), inhibin beta C chain (INHBC), inhibin beta E (INHBE), left-right determination factor 1 (LEFTY1), left-right determination factor 2 (LEFTY2), myostatin (MSTN), NODAL, neurturin (NRTN), persephin (PSPN), fibroblast growth factor 1 (FGF1), fibroblast growth factor 2 (FGF2), fibroblast growth factor 3 (FGF3), fibroblast growth factor 4 (FGF4), CBFA1/RUNX2, OSTERIX, SRY-box containing gene 9 (SOX9), Interleukin 1 Receptor Antagonist (IL1RA), Interleukin 10 (IL10), Chondroitinase ABC and Neurotrophin-3 (NT3), hepatocyte growth factor (HGF), granulocyte colony-stimulating factor (G-CSF), macrophage colony-stimulating factor (M-CSF), interleukin 6 (IL6), brain-derived neurotropic factor (BDNF), neurotrophin-4 (NT-4), nerve growth factor (NGF), insulin-like growth factor 1 (IGF-1), insulin-like growth factor-2 (IGF-2), fibroblast growth factor 21 (FGF21), or human growth hormone (HGH).

In certain implementations, the cargo can include a nucleic acid, for instance nucleic acids encoding antigenic polypeptides. In some embodiments, the vaccine can include a nucleic acid sequence (e.g., mRNA) encoding a human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, herpes simplex virus 1 (HSV1) antigenic polypeptide, herpes simplex virus 2 (HSV2) antigenic polypeptide, poxvirus (e.g., smallpox, monkeypox) antigenic polypeptide, cytomegalovirus antigenic polypeptide, Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide, Japanese encephalitis antigenic polypeptide, yellow fever antigenic polypeptide, Rift Valley fever antigenic polypeptide, hepatitis A, B, C, D, and E virus antigenic polypeptide, or coronaviruses (e.g., MERS-CoV, SARS-CoV, SARS-CoV2, HCoV-OC43, HCoV-229E, HCoV-NL63, HCoV-NL, HCoV-NH, HCoV—HKU1) antigenic polypeptide, African swine fever (ASF) antigenic polypeptide, foot-and-mouth disease virus (FMDV) antigenic polypeptide, feline herpesvirus-1/feline viral rhinotracheitis antigenic polypeptide, canine distemper antigenic polypeptide or any combination thereof. In some embodiments, the vaccine can include a nucleic acid sequence (e.g., mRNA) encoding monkeypox antigenic polypeptide, herpes simplex virus 1 (HSV1) antigenic polypeptide, herpes simplex virus 2 (HSV2) antigenic polypeptide, coronavirus (e.g., SARS-CoV2) antigenic polypeptide, or any combination thereof.

In some implementations, the cargo can include other antigens, for example killed, inactivated, attenuated, or modified live bacteria, viruses, or parasites. In certain implementations the compositions can include one or more cells, for example cancer cells.

In other implementations, the cargo may include an antigen derived from a virus such as from HIV-1, (such as tat, nef, gp120 or gp160), human herpes viruses, such as gD or derivatives thereof or Immediate Early protein such as ICP27 from HSV1 or HSV2, cytomegalovirus ((esp. Human)(such as gB or derivatives thereof), Rotavirus (including live-attenuated viruses), Epstein Barr virus (such as gp350 or derivatives thereof), Varicella Zoster Virus (such as gpl, II and IE63), or from a hepatitis virus such as hepatitis B virus (for example Hepatitis B Surface antigen or a derivative thereof), hepatitis A virus, hepatitis C virus and hepatitis E virus, or from other viral pathogens, such as paramyxoviruses: Respiratory Syncytial virus (such as F and G proteins or derivatives thereof), parainfluenza virus, measles virus, mumps virus, human papilloma viruses (for example HPV6, 11, 16, 18, etc.), flaviviruses (e.g., Yellow Fever Virus, Dengue Virus, Tick-borne encephalitis virus, Japanese Encephalitis Virus) or Influenza virus (whole live or inactivated virus, split influenza virus, grown in eggs or MDCK cells, or whole flu virosomes (as described by Gluck, Vaccine, 1992, 10, 915-920) or purified or recombinant proteins thereof, such as HA, NP, NA, or M proteins, or combinations thereof).

In certain implementations, the cargo can include an antibody, for example a monoclonal antibody. In certain implementations the cargo can include one of adalimumab, infliximab, basiliximab, daclizumab, omalizumab, aducanemab, lecanemab, bapineuzmab, solanezuab, gautenerumab, crenezumab, aducanemab, or donanemab.

In certain implementations, the cargo can include etanercept.

In certain implementations, the cargo can include a cytokine. In certain implementations, the composition can include interleukin 11 (IL-11), interferon beta, granulocyte macrophage colony-stimulating factor (GM-CSF), or erythropoietin.

### EXAMPLES

The following examples are for the purpose of illustration of the invention only and are not intended to limit the scope of the present invention in any manner whatsoever.

Furfurylamine, 5-Methyl-2-furanmethanamine, and glutaric anhydride were purchased from Sigma-Aldrich. Dimethyl acetylenedicarboxylate (DMAD), 3-Chloroperoxybenzoic acid (mCPBA), N,N′-Dicyclohexylcarbodiimide (DCC), 4-Dimethylaminopyridine (DMAP), and hydrated p-toluenesulfonic acid (PTSA) were obtained from Acros Organics, and ethyl 4,4,4-trifluoro-2-butynoate was supplied by Petra Research. These chemicals were used as received. Dichloromethane (DCM), toluene, hexane, ethyl acetate, methanol, and anhydrous dimethylformamide (DMF) were supplied by VWR and used as received. Water was purified using a Millipore Milli-Q system. Dextran (Mₙ=20 kDa), denoted as dextran 20 k, and lithium chloride were purchased from Alfa Aesar and dried in a vacuum oven at 80° C. overnight before use. 4-(Dimethylamino)pyridinium 4-toluenesulfonate (DPTS) was synthesized from an equal molar reaction of DMAP and PTSA, and recrystallized from toluene. Divalent PEG-thiols (PEG-2-SH) and tetravalent PEG-thiols (PEG-4-SH) were purchased from JenKem Technologies and stored in a nitrogen-purged glove box. The thiol content was periodically assessed using standard Ellman's Assay and found to be 90% and 95% respectively. Dulbecco's phosphate-buffered saline (1×PBS) containing 10 mM phosphate was supplied by Corning and the buffer pH was adjusted dilute HCl and NaOH. Qβ virus-like particles (VLPs) enveloping red fluorescent proteins (ex/em=585 nm/610 nm) were prepared according to a previous publication, and polystyrene (PS) beads of different sizes (15 nm and 30 nm) and fluorescence (ex/em=460 nm/500 nm, 545 nm/566 nm) were supplied by Lab261. Both VLP and PS particles were dissolved in PBS (1×, pH 6.0) at a concentration of 2.0 mg/mL.

Mouse Immunoglobulin G (IgG), a prototypical monoclonal antibody (mAb, MW ^{~}150 kDa) was purchased from Thermo Fisher Scientific. Cyanine 647 succinimidyl ester dye (ex/em=650 nm/665 nm) obtained from Biotium was used label the IgG via the standard N-Hydroxysuccinimide (NHS) ester reaction. Following standard purification procedures, the dye-labeling efficiency was quantified as approximately one dye molecule per IgG molecule. This was validated through standard fluorescence calibration and liquid chromatography-mass spectrometry (LC-MS) analysis. The IgG particles were dissolved in PBS (1×, pH 6.0) at a concentration of 2.0 mg/mL.

### Example 1: Synthesis of OND Linkers

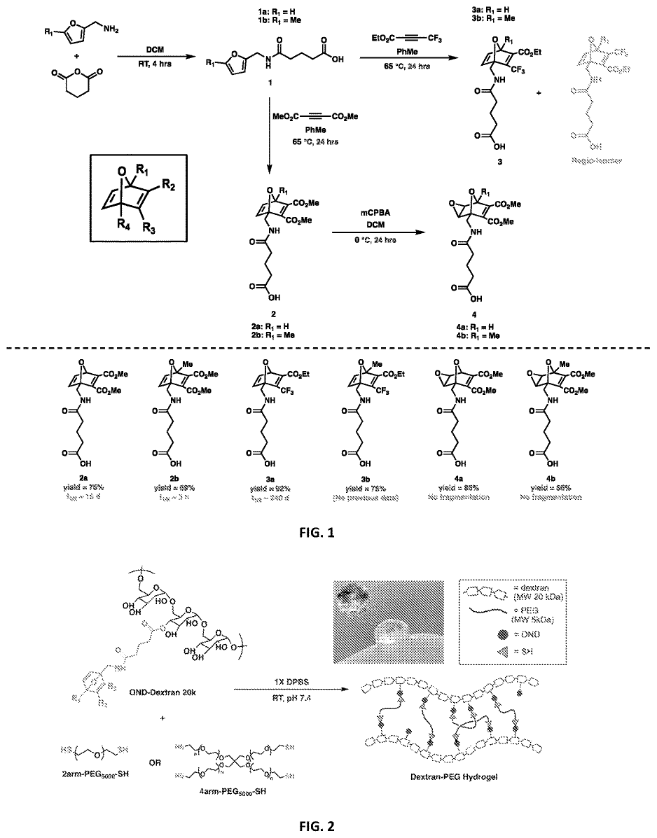

Three classes of OND linkers with varying fragmentation half-lives were synthesized through [4+2]cycloaddition of furans with alkynes (as shown in FIG. 1). First, a solution of the precursor furan (either furfurylamine or 5-Methyl-2-furanmethanamine, 1 equiv) in DCM (1 M) was slowly mixed with a solution of glutaric anhydride (1.7 equiv) in DCM (1.5 M) at room temperature to synthesize furan molecule 1, denoted as Furan 1. After 4 h, the resulting mixture was filtered, and the product was obtained by washing with cold DCM. Furan 1 then underwent reactions with DMAD and ethyl 4,4,4-trifluoro-2-butynoate (both 1.5 equiv to Furan 1) in toluene (1 M of Furan 1) with a few drops of methanol to produce OND 2 and OND 3, respectively, at 65° C. for 24 h. OND 2 was obtained by triturating the resulting solution against ethyl ether, while OND 3 was purified using Biotage flash chromatography in a hexane/ethyl acetate solvent system. It is worth noting that OND 3 was comprised of a mixture of two regio-isomers due to the asymmetry of ethyl 4,4,4-trifluoro-2-butynoate. The mixture was used in the subsequent steps without further purification, as the ratio of the isomers was consistent from batch to batch. Additionally, OND 4 was produced from the epoxidation of OND 2, using mCPBA (1.5 equiv to OND 2) as the oxidant, adding to the solution of OND 2 in DCM (0.1 M). The reaction was carried out on the ice for 24 h, and the product was purified by column (ethyl acetate/methanol).

The ONDs synthesized previously were attached to dextran 20 k sidechains by ester linkages. After being dried under vacuum overnight, dextran (average molecular weight 20 kDa, 334 mg, 2.0 mmol glucose units, 1 equiv) and LiCl (600 mg, 14 mmol, 7 equiv) were added to 30 mL of anhydrous DMF and stirred at 90° C. for 30 minutes, resulting in a clear solution, which was then cooled to 0° C. This cooled solution was mixed with OND (0.4 equiv), DCC (518 mg, 2.5 mmol, 1.25 equiv), and DPTS (77.5 mg, 0.27 mmol, 0.14 equiv), and stirred on ice for 3 days. The crude product was then precipitated in 150 mL of acetone and further purified by standard dialysis (MW cutoff 3500 Da) against water for 2 days. Finally, the solution was filtered through a 0.2 μm syringe filter, frozen, and lyophilized to yield a white, fluffy dextran-OND product. Repeated preparations showed that the degree of substitution (DS), or the number of ONDs attached per glucose unit, was 14-16%, as characterized by 1H NMR analysis.

### Example 2: Preparation of Cargo-Laden Hydrogels

Hydrogels were prepared by first dissolving dextran-OND and PEG-SH materials separately in PBS buffers containing the desired cargo. The two solutions were combined in a 1:1 ratio of OND to thiol, resulting in gels each containing 10 wt % of polymers and measuring 100 μL in size (FIG. 2). Previous studies have demonstrated that increasing the buffer concentration and pH can accelerate gelation. To facilitate subcutaneous injection, gelation was tested at 6.0 pH in 1×PBS, either at room temperature or 4° C., and confirmed by an inversion test of the vials. Both VLPs (30 nm) and PS beads (15 nm, 30 nm) were chosen as cargos.

The cargo-loaded hydrogels were immersed in 1 mL of blank PBS (1×, pH 7.4) as the releasing buffer. The cargo loading capacity was determined by measuring the fluorescence of the supernatant immediately after gelation. The same measurement was taken every 12 hours to generate the in vitro cargo release curve while the hydrogels were incubated at 37° C. At each time point, 100 μL of the releasing buffer was collected and replaced with the same amount of fresh buffer.

The rheological properties of the hydrogels were characterized using a TA instrument HR-2 Discovery Hybrid Rheometer, equipped with a 40 mm diameter parallel plate geometry. Solutions of dextran-OND and PEG-SH in PBS, as described above, were quickly mixed and deposited on the substrate. The spindle was lowered onto it to achieve a gap of 350 μm, and a solvent trap (TA instrument) was used to prevent evaporation during the experiment. The entire experiment was conducted at room temperature. During gelation, the storage modulus (G′) and loss modulus (G″) were monitored with time at a frequency of 1 Hz and a shear strain of 5%. Once the G′ and G″ values had reached a plateau, oscillatory frequency sweeps were performed from 0.1 to 100 Hz at a fixed strain of 5%, and oscillatory shear strain sweeps were carried out from 1% to 100% at a fixed frequency of 1 Hz.

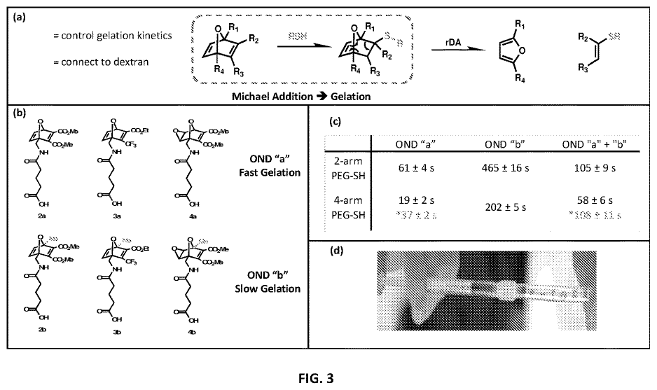

The Michael addition reaction between OND molecules and thiols leads to gelation, and the kinetics of this process are controlled by the R¹ substitution (refer to FIG. 3a). ONDs without any substitution at R¹ are denoted as OND “a” (2a, 3a, 4a), while those with a CH₃ substitution are denoted as OND “b” (2b, 3b, 4b) (refer to FIG. 3b). Group “b” exhibits a slower Michael addition rate than group “a” due to the increased steric hindrance at the location of the addition reaction caused by CH₃ at R¹, which slows down the reaction kinetics. As shown in FIG. 1c, gels fabricated using OND “a” materials formed much faster than those fabricated using OND “b” materials. The gels containing half OND “a” and half OND “b” showed intermediate gelation speed. Additionally, gels made of PEG-4-SH resulted in a shorter gelation time than those made of PEG-2-SH, as the higher valency of thiols increases the likelihood of reacting with ONDs.

The injectability of the gel materials was validated using a clinically validated syringe-to-syringe transfer technique, which involved using separate syringes for dextran-OND and PEG-SH connected by a double female luer lock adapter (Air-Tite), as shown in FIG. 3d. To prevent the needle from clogging if gelation occurs too quickly, certain formulations (shown in FIG. 3c in *blue color) were cooled to 4° C. to slow down the gelation process (as Michael addition kinetics are slower at lower temperatures). As a result, most formulations took more than one minute to gel, providing ample time to mix the two solutions before injection.

OND 3 exhibited a significantly slower rDA fragmentation rate (with a half-life over a month) than OND 2 (with a half-life ranging from hours to days) upon thiol-Michael addition. Importantly, the CH₃ substitution at the R¹ position also significantly influenced the kinetics of rDA, which is the step that causes gel degradation and cargo release. Thus, OND 2b and 3b are faster degradable versions of the linker OND 2a and 3a, respectively. OND 4, the epoxidation product of OND 2, could not fragment due to the lack of bond-rearrangement capacity in the ring. The cargo was physically entrapped in the gel network during gelation and slowly diffused out during the gel degradation. A delayed-burst release curve was achieved due to the aqueous hydrolysis of the ester bonds connecting ONDs and the dextran backbone. Other release profiles (such as first-order, linear, and exponentially increasing) relied on OND rDA fragmentation triggered by reacting with thiol nucleophiles. The cargo loading capacity of each gel was read from the release curve at time 0, and it was found that all formulations had successfully entrapped more than 90% of the cargo into the gel.

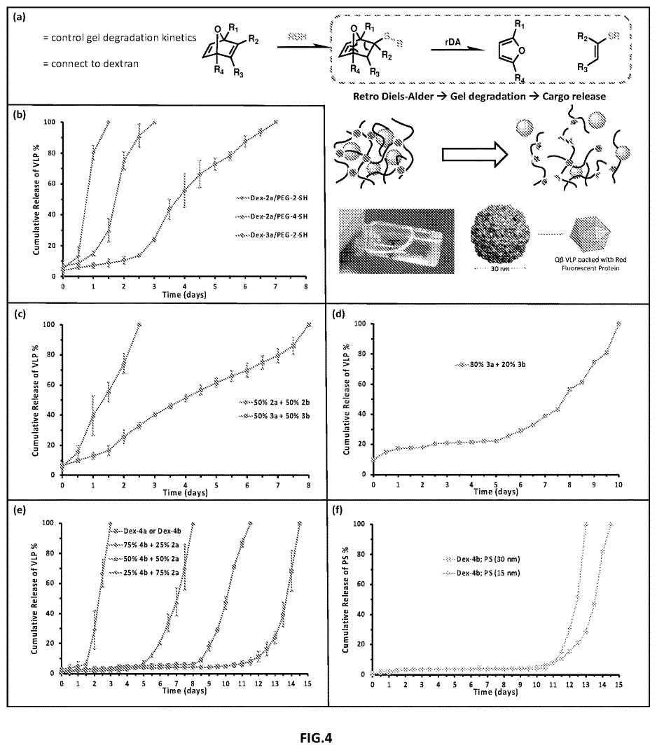

A biphasic release profile can be observed in hydrogel release systems, particularly when cargo size exceeds the initial gel mesh dimensions. It consists of two distinct phases: an initial lag phase characterized by minimal or no cargo release, followed by a first-order diffusion-based release phase during which a substantial portion of the cargo is rapidly released. The lag phase arises from the cargo's restricted mobility within the gel network, as an insufficient number of broken linkers limit available space for diffusion. Once this barrier is overcome, the cargo is released more swiftly, culminating in the burst release phase. FIG. 4b demonstrates that hydrogel formulations H1 (dex-2a/PEG-2-SH), H2 (dex-2a/PEG-4-SH), and H3 (dex-3a/PEG-2-SH) exhibit this profile. H3, with a longer lag phase and more protracted release compared to H1, is due to the considerably extended half-lives of OND 3a linkers relative to OND 2a, causing the 3a linker-based gel to require more time to reach the cargo release threshold. Comparing H2 and H1, both gels utilize 2a linkers; however, H2 possesses a higher crosslinking density than H1 (tetravalent PEG-thiols vs. divalent PEG-thiols), which accounts for the prolonged release observed from the H2 gel, as a greater number of linkers must be cleaved to achieve the cargo release threshold. Thus, to achieve a longer window, tetravalent PEG-thiols (PEG-4-SH) were used in all the following studies.

Hybrid hydrogels containing a combination of OND linkers exhibited intriguing release profiles. As depicted in FIG. 4c, hydrogel H4 (50% 2a+50% 2b), composed of equal parts dextran-OND 2a and dextran-OND 2b, achieved a linear release of VLPs in 2.5 days. Meanwhile, hydrogel H5 (50% 3a+50% 3b), consisting of a 50/50 blend of dextran-OND 3a and dextran-OND 3b, accomplished linear release within 8 days. Zero-order release kinetics were attained by incorporating specific ratios of rapidly and slowly fragmenting linkers-2a with its faster-degrading counterpart 2b, and 3a with its faster-degrading counterpart 3b. Notably, gels composed exclusively of OND 2b and OND 3b fully degraded in 12 and 24 hours, respectively. By including 2b and 3b linkers, the initial lag phase observed in FIG. 4b was effectively eliminated, as linker fragmentation was accelerated from the beginning. As a result, these hybrid hydrogels successfully demonstrated zero-order release kinetics, a feat unattainable by traditional hydrogels.

FIG. 4d demonstrates an exponentially increasing release of VLPs from hydrogel H6 (80% 3a+20% 3b), which comprises an 80/20 blend of dextran-OND 3a and dextran-OND 3b. In comparison to H5, H6 contains a smaller proportion of 3b linkers, resulting in a slower degradation rate and extended cargo release, totaling 10 days. The accelerated release rate observed after day 7 could be attributed to the hydrolysis of ester bonds, as the OND linkers are connected to the dextran backbone via esterification. These hydrolysable ester bonds contribute to a cumulative effect on gel degradation. The gel also exhibits degradation, albeit over a more extended period, when using non-fragmentable OND linkers.

The discovery of the delayed-burst release mechanism was serendipitous, as the original hypothesis posited that hydrogels made of non-fragmentable linkers (OND 4a or 4b) would not degrade, and thus no release should be observed. Contrary to expectations, the in vitro release study revealed that gel H7 (dex-4a or 4b), consisting of either dextran-4a or dextran-4b, exhibited a consistent delayed-burst release beginning on day 11 and concluding on day 14.5. This outcome refuted the initial hypothesis and suggested the presence of another factor contributing to gel degradation besides OND rDA reactions. This factor was determined to be ester bond hydrolysis (connecting OND to dextran), which was confirmed by incubating the gel in buffers with varying pH levels, given that ester hydrolysis is highly pH-dependent. The gel incubated in PBS pH 8.4 fully collapsed in 9 days, while the gel incubated in PBS pH 6.4 remained stable for over 50 days. This result demonstrates that H7's degradation is governed by ester bond hydrolysis.

This finding confirms that the hydrogels synthesized in this study underwent degradation due to dynamic meshwork rearrangement rather than hydrogel swelling, another common factor causing mesh size increase and cargo release. This conclusion is supported by the fact that release from swelling-dependent hydrogels should not be pH-dependent. Moreover, the other three hydrogels in FIG. 4e are hybrid gels containing varying ratios of the fragmentable OND linker 2a. It was observed that gels with higher 2a content collapsed more rapidly than those with less or no 2a linkers. This observation is logical, as a greater number of degradable linkers would accelerate the increase in gel mesh size, creating sufficient space for cargo release.

Furthermore, the H7 delayed-burst release gel was employed to investigate the release of differently sized cargo from a single gel. Polystyrene (PS) beads of 15 nm and 30 nm were utilized as model cargo. As depicted in FIG. 4f, the curve generated from measuring the release of 30 nm PS beads exhibited no difference from the corresponding VLP release curve in FIG. 4e, as they share the same size. However, the smaller 15 nm PS beads diffused out of the gel network at a faster rate compared to the 30 nm beads. This is attributable to the fact that fewer linkers need to be cleaved to allow the smaller cargo to diffuse out of the gel network, and they can diffuse more rapidly than the larger cargo upon reaching the onset threshold.

### Example 3—Synthesis of OND Linkers and OND-Functionalized Dextran Polymers

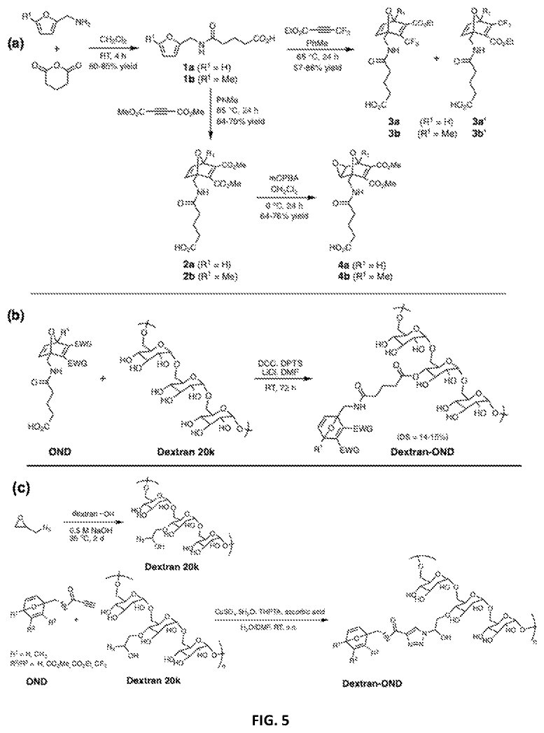

Three classes of OND linkers with varying fragmentation half-lives were synthesized through [4+2]cycloaddition of furans with alkynes (as shown in FIG. 5). First, a solution of the precursor furan (either furfurylamine or 5-Methyl-2-furanmethanamine, 1 equiv) in DCM (1 M) was slowly mixed with a solution of glutaric anhydride (1.7 equiv) in DCM (1.5 M) at room temperature to synthesize furan molecule 1, denoted as Furan 1. After 4 h, the resulting mixture was filtered, and the product was obtained by washing with cold DCM. Furan 1 then underwent reactions with DMAD and ethyl 4,4,4-trifluoro-2-butynoate (both 1.5 equiv to Furan 1) in toluene (1 M of Furan 1) with a few drops of methanol to produce OND 2 and OND 3, respectively, at 65° C. for 24 h. OND 2 was obtained by triturating the resulting solution against ethyl ether, while OND 3 was purified using Biotage flash chromatography in a hexane/ethyl acetate solvent system. It is worth noting that OND 3 was comprised of a mixture of two regioisomers due to the asymmetry of ethyl 4,4,4-trifluoro-2-butynoate. The mixture was used in the subsequent steps without further purification, as the ratio of the isomers was consistent from batch to batch. Additionally, OND 4 was produced from the epoxidation of OND 2, using mCPBA (1.5 equiv to OND 2) as the oxidant, adding to the solution of OND 2 in DCM (0.1 M). The reaction was carried out on the ice for 24 h, and the product was purified by column (ethyl acetate/methanol).

The ONDs synthesized previously were attached to dextran 20 k sidechains by ester linkages. After being dried under vacuum overnight, dextran (average molecular weight 20 kDa, 334 mg, 2.0 mmol glucose units, 1 equiv) and LiCl (600 mg, 14 mmol, 7 equiv) were added to 30 mL of anhydrous DMF and stirred at 90° C. for 30 minutes, resulting in a clear solution, which was then cooled to 0° C. This cooled solution was mixed with OND (0.4 equiv), DCC (518 mg, 2.5 mmol, 1.25 equiv), and DPTS (77.5 mg, 0.27 mmol, 0.14 equiv), and stirred on ice for 3 days. The crude product was then precipitated in 150 mL of acetone and further purified by standard dialysis (MW cutoff 3500 Da) against water for 2 days. Finally, the solution was filtered through a 0.2 μm syringe filter, frozen, and lyophilized to yield a white, fluffy dextran-OND product. Repeated preparations showed that the degree of substitution (DS), or the number of ONDs attached per glucose unit, was 14-16%, as characterized by ¹H NMR analysis.

To prepare non-hydrolysable dextran with triazole linkages, ONDs linkers with a propiolamide group were synthesized. First, a solution of 2-ethoxy-1-ethoxycarbonyl-1,2-dihydroquinoline (EEDQ; 1 equiv) coupling reagent and propiolic acid (1 equiv) in DMF was stirred under inert atmosphere for 15 minutes then a precursor furan (either furfuryl amine or 5-methyl-2-furanmethaneamine, 1 equiv) was added slowly at 0° C. The reaction mixture was allowed to warm up to room temperature and stirred overnight. The reaction crude was diluted in ethyl acetate, washed with water and brine, dried over sodium sulfate, and concentrated to obtain the crude product which was purified by silica gel chromatography to yield a yellow oil. The resulting furans underwent [4+2]cycloaddition with either DMAD, DEAD, or ethyl 4,4,4-trifluoro-2-butynoate as described above to obtain alkyne-containing OND linkers.

Non-hydrolysable dextran polymers were prepared by epoxide ring-opening followed by click reactions with alkyne-containing OND linkers. To a solution of dextran (200 mg) in 1.23 ml of 0.5 N NaOH was added 2-(azidomethyl)oxirane (0.5-3 equiv to a unit of glucose monomer) and stirred at 35° C. for 2 days. The product was precipitated in 2-propanol, filtered, and washed with 2-propanol. The filtered polymer was dialyzed against water for 2 days and lyophilized, producing a white solid with 12-69% azide functionalization (relative to the glucose units), as characterized by ¹H NMR. The resulting polymer was conjugated with OND linkers by the azide-alkyne click reaction. First, a solution of copper sulfate pentahydrate (1 equiv to the azide unit on dextran) and sodium ascorbate (5 equiv) was stirred in water (1 mL) for a couple of minutes then tris-hydroxypropyltriazolylmethylamine (THPTA; 3 equiv) in water (1 mL) was added and stirred until the solution became colourless to yellow. To the reaction solution, the azide-functionalized dextran (50 mg) in water (0.5 mL) and OND in DMF (1 mL) were added and a freeze-thaw-cycle was conducted three times then stirred overnight under Ar. The reaction mixture was dialyzed against water for 2 days and lyophilized to obtain a white solid.

### Example 4—Preparation of OND-Hydrogels with Entrapped Cargo

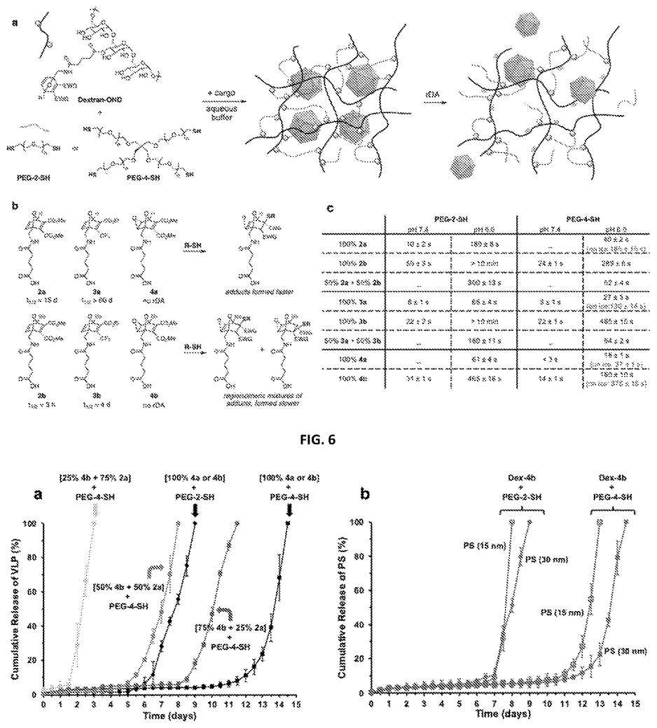

Hydrogels were prepared by first dissolving dextran-OND and PEG-SH materials separately in PBS buffers containing the desired cargo. The two solutions were combined in a 1:1 ratio of OND to thiol, with the overall concentration of polymers varying from 2.5 wt % to 30 wt % depending on the size of the cargo and the desired release properties (FIG. 6a). Each gel was made in 100 μL sized volume, although this can be changed at will. Previous studies have demonstrated that increasing the buffer concentration and pH can accelerate gelation. To facilitate subcutaneous injection, gelation was tested at 6.0 pH in 1×PBS, either at room temperature or 4° C., and confirmed by an inversion test of the vials. Fluorescent VLPs (30 nm diameter), polystyrene beads (15 nm, 30 nm diameter), monoclonal antibodies (150 kDa molecular weight), and non-thiol modified sfGFP (30 kDa molecular weight) were chosen as cargos. None of these cargo particles display reactive thiols, so the entrainment is entirely physical. Each gel contained cargos at the concentration of ^{~}2.0 mg/ml; this can be changed at will.

The cargo-loaded hydrogels were immersed in 1 mL of blank PBS (1×, pH 7.4) as the releasing buffer. The cargo loading capacity was determined by measuring the fluorescence of the supernatant immediately after gelation. The same measurement was taken every 12 hours or 24 hours to generate the in vitro cargo release curve while the hydrogels were incubated at 37° C. At each time point, 100 μL of the releasing buffer was collected and replaced with the same amount of fresh buffer.

The rheological properties of the hydrogels were characterized using a TA instrument HR-2 Discovery Hybrid Rheometer, equipped with a 40 mm diameter parallel plate geometry. Solutions of dextran-OND and PEG-SH in PBS, as described above, were quickly mixed and deposited on the substrate. The spindle was lowered onto it to achieve a gap of 350 μm, and a solvent trap (TA instrument) was used to prevent evaporation during the experiment. The entire experiment was conducted at room temperature. During gelation, the storage modulus (G′) and loss modulus (G″) were monitored with time at a frequency of 1 Hz and a shear strain of 5%. Once the G′ and G″ values had reached a plateau, oscillatory frequency sweeps were performed from 0.1 to 100 Hz at a fixed strain of 5%, and oscillatory shear strain sweeps were carried out from 1% to 100% at a fixed frequency of 1 Hz.

The Michael addition reaction between OND molecules and thiols leads to gelation, and the kinetics of this process is strongly affected by bridgehead substitution (R¹ in FIG. 6a); the gelation speed was relatively fast for R¹═H (structure 2a, 3a, 4a) and slow for R¹═CH₃ (2b, 3b, 4b), presumably due to steric hinderance. Gels containing equal molar amounts of OND “a” and OND “b” showed intermediate gelation speed (FIG. 6c). The “b” group also gave rise to regioisomeric mixtures of adducts, which we characterized and used without separation (FIG. 6b). Additionally, gels made of PEG-4-SH resulted in a shorter gelation time than those made of PEG-2-SH, as the higher valency of thiols increases the likelihood of reacting with ONDs as well as the crosslinking density in the gel.

The injectability of the gel materials was validated by (1) pre-mixing the dextran-OND and PEG-SH materials and then drawing the mixture into a syringe for injection, or (2) through the use of a standard syringe-to-syringe transfer technique, which involved using separate syringes for dextran-OND and PEG-SH connected by a double female luer lock adapter (Air-Tite). To prevent the needle from clogging if gelation occurs too quickly, certain formulations (shown in FIG. 6c) were cooled to 4° C. (i.e., “on ice”) to slow down the gelation process (as Michael addition kinetics are slower at lower temperatures). As a result, most formulations took more than one minute to gel, providing ample time to mix the two solutions before injection. All formulations gelation times greater than 15 seconds successfully entrapped more than 90% of the cargo into the gel.

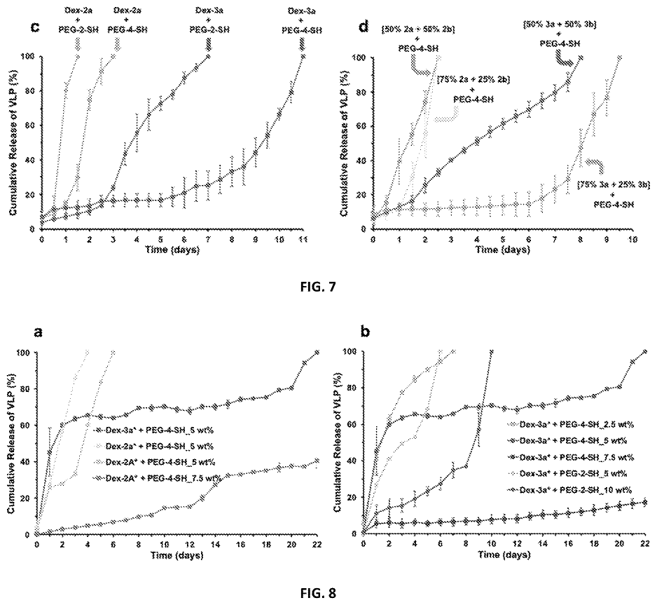

In this example, depicted in FIG. 7, two chemical processes are responsible for gel degradation and cargo release. One is hydrolysis of the ester bond connecting the dextran polymer to the OND unit. This process is unaffected or minimally affected by the OND group. The second process responsible for hydrogel degradation and cargo release is the retro-Diels-Alder (rDA) reaction (fragmentation) of the OND-thiol conjugate. Different rDA half-lives are presented in FIG. 6b, where OND 3a demonstrates a considerably slower rDA fragmentation rate compared to OND 2a. Consequently, gels formed with Dex-3a are inherently more stable, resulting in prolonged release profiles compared to those fabricated with Dex-2a, as illustrated in FIG. 7c. Notably, the methyl substitution at the R1 position (FIG. 6a, 6b) markedly impacts the kinetics of the rDA reaction. Thus, OND 2b and 3b serve as more rapidly degrading variants of the linker ONDs 2a and 3a, respectively. FIG. 7d displays the release curves obtained by combining slow- and fast-degrading OND linkers, such as 2a with 2b and 3a with 3b, in various proportions, achieved distinct linear and extended release of VLPs.

The same principle applies to non-hydrolysable hydrogels (FIG. 8), which were fabricated using dextran-OND polymers synthesized as per FIG. 5c. The ‘*’ symbol in this figure denotes dextran-OND connected via a non-hydrolysable triazole linkage, indicating that gel degradation and cargo release are due exclusively to OND rDA fragmentation. Gels composed of Dex-3a* showed more extended-release profiles compared to those made with Dex-2a* and Dex-2A* (where ‘A’ indicates CO₂Et at both the R² and R³ positions in FIG. 5c), because OND 3a possesses longer rDA half-lives than OND 2a (or 2A). Additionally, gels with higher PEG valences and higher polymer weight percentages (FIG. 8b) demonstrated increased stability due to a higher degree of crosslinking.

OND 4, the epoxidation product of OND 2, cannot undergo rDA fragmentation due to the lack of bond-rearrangement capacity in the ring. When OND 4 was used, delayed-burst release was observed at a time point determined by the crosslinking density employed, due to the aqueous hydrolysis of the ester bonds connecting ONDs and the dextran backbone. (This mechanism was confirmed by measurement of gel integrity in buffers of different pH, in which gels were found to collapse faster in more basic media (pH 6.4>50 days; pH 8.4=9 days). Replacing some of the epoxide-OND with increasing amounts of rDA-active OND 2a progressively shortened the cargo retention period, but the burst release pattern was unaltered (FIG. 7a). The compiled data indicates that the strategic combination of rDA and non-rDA versions of ONDs in precise ratios can finely adjust the delayed-burst release of VLPs at targeted time intervals. Other release profiles (such as first-order, linear, and exponentially increasing) were observed using OND rDA fragmentation triggered by reacting with thiol nucleophiles. Examples of all these release profiles shown in FIGS. 7 and 8 demonstrate that VLP release curves can be significantly modulated by utilizing single or combinations of diverse ONDs.

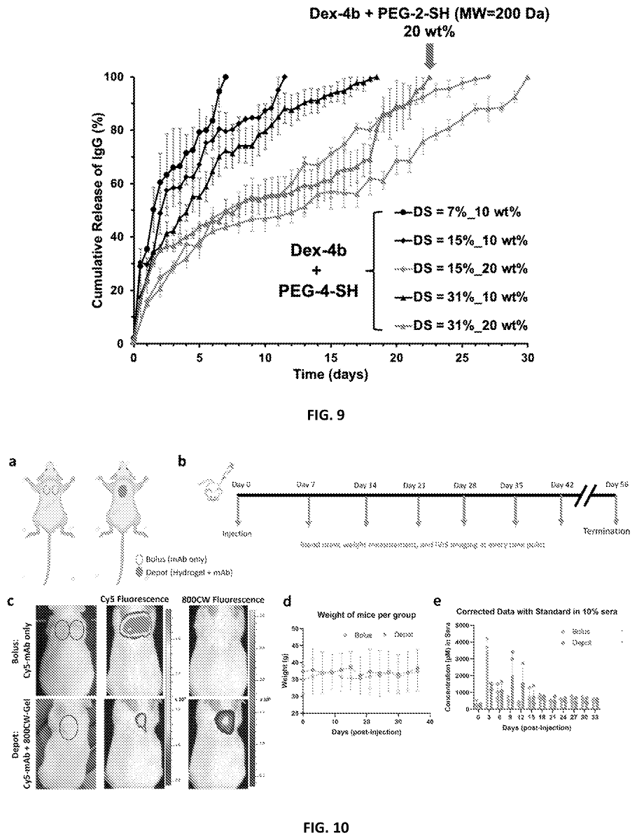

An important parameter in the cargo release function of these hydrogels is the size of the cargo molecule. This is illustrated in comparison between the release profiles shown in FIG. 7a,b (Dex-4b+PEG-4-SH) and FIG. 9), which employ the same hydrogel formulation but different cargo molecules (virus-like particles and green fluorescent protein, respectively). The former is released in a burst profile at approximately two weeks, whereas the latter is released gradually in a different profile over the same period. Another example is provided in FIG. 7b, in which the Dex-4b+PEG-4-SH material releases polystyrene (PS) beads of two different sizes approximately 2-3 days apart. This is attributable to the fact that fewer linkages need to be cleaved to allow smaller cargos to diffuse out of the gel network, and they can diffuse more rapidly than the larger cargo upon reaching the onset threshold.

To validate the formation and use of this hydrogel as a controlled release system to enable extended release of cargo, eight mice were injected with 200 μg of Cy5-labeled mouse IgG subcutaneously (s.c.) at the dorsal flank between the shoulder blades (FIG. 10). Four of the mice received the dye labeled mAb cargo as two separate bolus injections and the other four mice received a single injection of the depot loaded with the dye-labeled mAb cargo (FIG. 10a). Blood was collected, and the mice were weighed and imaged using IVIS in vivo imaging system every three days for a total of 8 weeks (FIG. 10b). For the depot group, gels formed s.c. within 30 seconds following injection. Gelation was validated by visual observation of gelled materials in the syringe and by gently palpation of the skin at the injection site. The presence of Cy5-labeled cargo and the IR800CW-labeled hydrogel were visualized using PerkinElmer IVIS Lumina imaging system. For both groups, clear fluorescence signal from the cargo was observed at injection site, however fluorescent signal from the depot was only observed in the depot group (FIG. 10c). Throughout the eight weeks of this study, mice weight remained steady, and no significant difference was observed between groups (repeated measures Anova for statistical analysis) indicating that the material was well tolerated (FIG. 10d). Following collection, blood was centrifuged in a collection tube containing SST to enable separation of sera from red blood cells. Cargo concentration in sera was determined using 10× dilutions in PBS (1×, pH 7.4) for all groups and time points, where the standard curve was prepared from dye-labeled cargo in 10% serum (FIG. 10e). Initial pharmacokinetic analysis indicates that the depot group demonstrated higher concentrations of cargo in sera at later time points, thus validating the extended-release capability of this OND-hydrogel system in vivo.

### Example 5—Preparation of OND-Hydrogels with Covalently Attached Cargo

Cargo molecules may be covalently attached to the hydrogel polymer (such as dextran) via OND connections in one of two general ways: (1) by attachment of the OND connector to the polymer for reaction with a nucleophilic group (thiol or amine, for example) on the cargo, or (2) by attachment of the OND connector to the cargo for reaction with a nucleophilic group (thiol or amine, for example) on the polymer. This example is of the first category. In both cases, release of the cargo from the hydrogel is also dependent on noncovalent entrapment which is modulated by the cleavage of bonds crosslinking the hydrogel components.

Superfolder green fluorescent protein (sfGFP) was chosen as the cargo to demonstrate proof-of-concept for the release of small proteins (MW ^{~}30 kD) in this hydrogel system. SfGFP was expressed at room temperature in BL21 DE3 Gold competent cells upon induction with 0.1M IPTG. Cells were lysed via sonication and protein was purified from the clarified lysate using TALON affinity columns according to standard immobilized metal affinity chromatography (IMAC) methods. Purified protein was modified to exhibit free surface thiols for incorporation into the OND-hydrogels by reaction with Traut's reagent for 1 hour at room temperature. The extent of thiolation was quantified using an Ellman's assay and validated with liquid chromatography mass spectrometry (LCMS).

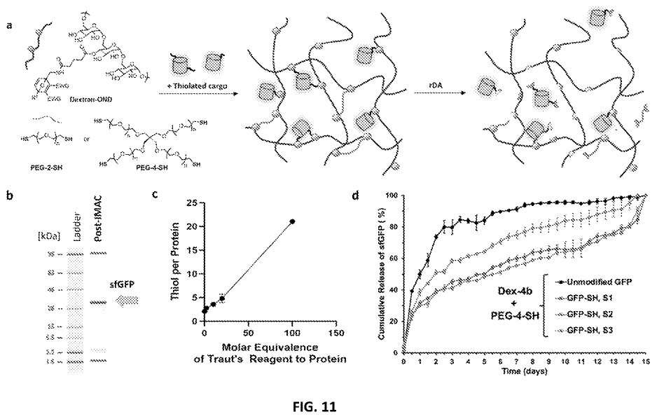

Hydrogels with cargo covalently attached were prepared in a manner similar to those with physically entrapped cargo, with the distinction being how the thiol-functionalized cargo was combined with the dextran-OND and/or PEG-SH components. For example, as depicted in FIG. 11, sample S1 was prepared by dissolving both PEG-SH and dextran-OND in a buffered solution containing thiol-functionalized GFP. Sample S2 was produced by initially mixing thiol-functionalized GFP cargo exclusively with PEG-SH before reacting with dextran-OND to form the gel. Conversely, sample S3 was created by premixing the thiol-containing cargo solely with dextran-OND before the subsequent reaction with PEG-SH to form the gel. Cargo release was quantified using the same method as before, with sfGFP being progressively released as shown in FIG. 11d. The data indicates that samples S1 and S3 exhibited enhanced cargo retention compared to S2. This is likely due to the improved opportunity for the thiol groups on the cargo to react with the ONDs on the dextran polymer, thereby increasing the likelihood of the cargo being securely anchored within the gel matrix and reducing diffusion out of the network.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

### Additional Implementations

1. A composition obtained by crosslinking a 7-oxanorbornene-functional polymer having the formula:

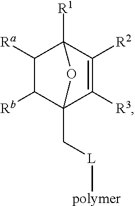

with a multi-valent nucleophile-terminated compound, wherein:
R^{a} and R^{b} together form an epoxide or double bond;
R¹ is selected from H, halo, C₁₋₄alkyl;
R² is selected from trifluoromethyl, cyano, nitro, C(═O)R^{c}, C(═O)OR^{c};
R³ is selected from trifluoromethyl, cyano, nitro, C(═O)R^{d}, C(═O)OR^{d};
wherein R^{c} and R^{d} are independently a C¹⁻⁴alkyl group, or R^{c} and R^{d} together form a ring;
L is a linker;
wherein the composition resists degradation when incubated at 23° C. in an aqueous solvent at pH between 6-6.8, but is degraded when incubated at 23° C. in an aqueous solvent at pH between 7.6-9.0.
2. The composition according to a preceding implementation, wherein the polymer has the formula:

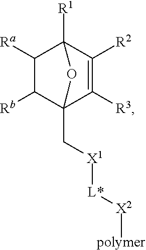

wherein:
X¹ is null, NH, O, NHC(═O), NHC(═O)NH;
L* is null, C₁₋₆alkyl, C₁₋₆heteroalkyl, alkheteroaryl;
X² is null NH, O, C(═O), NHC(═O), NHC(═O)NH.
3. The composition according to a preceding implementation, wherein L* comprises the product of a click-cycloaddition.
4. The composition according to a preceding implementation, wherein L* has the formula:

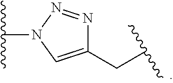 [SMILES: *Cc1cn(*)nn1]

5. The composition according to a preceding implementation, wherein R¹ is methyl.
6. The composition according to a preceding implementation, which is obtained by combining a multi-valent nucleophile-terminated compound with a first 7-oxanorbornene-functional polymer having the formula:

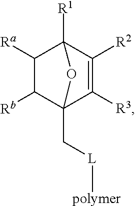

wherein:
R¹ is C₁₋₄alkyl; and
a second 7-oxanorbornene-functional polymer having the formula:

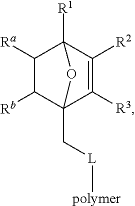

wherein:
R¹ is H.
7. The composition according to a preceding implementation, wherein the first and second 7-norbornene polymers are combined in a weight ratio from 10:1 to 1:10, from 10:1 to 1:1, from 10:1 to 5:1, from 5:1 to 1:1, from 2.5:1 to 1:1, from 1.5:1 to 1:1, from 1.25:1 to 1:1.25, from 1:1 to 1:10, from 1:5 to 1:10, from 1:1 to 1:5, from 1:1 to 1:2.5, or from 1:1 to 1:1.5.
8. The composition according to a preceding implementation, wherein the polymer comprises a polysaccharide, a polypeptide, a synthetic polymer, or a combination thereof.
9. The composition according to a preceding implementation, wherein the polymer comprises a cellulose, a hyaluronic acid, a dextran, an alginate, a chitosan, a pectin, a starch, a xanthan gum, or a combination thereof.
10. The composition according to a preceding implementation, wherein the polymer comprises carboxymethylcellulose, hydroxypropylcellulose.
11. The composition according to a preceding implementation, wherein the polymer comprises polyvinyl alcohol, poly(vinylpyrrolidone), poly(lactic acid), poly(lactic-co-glycolic acid) (PLGA), poly(meth)acrylic acid, poly(meth)acrylamide, poly(meth)acrylate, a copolymer thereof, or a combination thereof.
12. The composition according to a preceding implementation, wherein the polymer has a Mₙ from 5 kDa to 3,000 kDa, from 5 kDa to 100 kDa, from 5 kDa to 25 kDa, from 10 kDa to 25 kDa, from 20 kDa to 50 kDa, from 50 kDa to 150 kDa, from 100 kDa to 250 kDa, from 250 kDa to 1,000 kDa, from 1,000 kDa to 2,000 kDa, or from 2,000 kDa to 3,000 kDa.
13. The composition according to a preceding implementation, wherein the polymer comprises 7-oxanorbonene groups in an amount (per monomer unit) from 1-50%, from 1-25%, from 1-20%, from 1-10%, from 1-5%, from 5-10%, from 5-20%, from 10-15%, from 10-20%, from 10-25%, from 15-25%, or from 25-50%.
14. The composition according to a preceding implementation, wherein the multi-valent nucleophile-terminated compound comprises a multi-arm thiol.
15. The composition according to a preceding implementation, wherein the multi-valent nucleophile-terminated compound comprises a multi-arm PEG thiol.
16. The composition according to a preceding implementation, wherein the multi-valent nucleophile-terminated compound comprises a 2-arm PEG thiol, a 3-arm PEG thiol, a 4-arm PEG thiol, a 6-arm PEG thiol, an 8-arm PEG thiol, or a combination thereof.
17. The composition according to a preceding implementation, wherein the multi-valent nucleophile terminated compound has the formula:

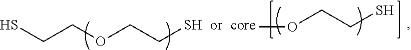

wherein the core has the formula:

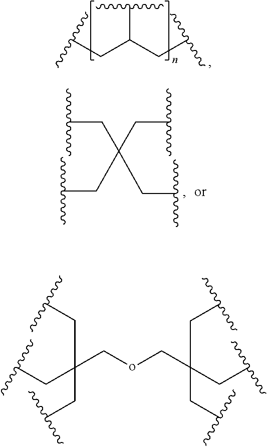

n is an integer from 1-6; and
the multi-valent nucleophile terminated compound has an average MW from 200-50,000 Da, from 200-2,500 Da, from 500-2,500 Da, from 1,000-5,000 Da, from 2,500-10,000 Da, from 5,000-10,000 Da, from 10,000-25,000 Da, from 10,000-50,000 Da, or from 25,000-50,000 Da.
18. The composition according to a preceding implementation, wherein the 7-oxanorbornene functionalized polymer and the multi-valent nucleophile-terminated compound are combined in a weight ratio from 10:1 to 1:10, from 10:1 to 1:1, from 10:1 to 5:1, from 5:1 to 1:1, from 2.5:1 to 1:1, from 1.5:1 to 1:1, from 1.25:1 to 1:1.25, from 1:1 to 1:10, from 1:5 to 1:10, from 1:1 to 1:5, from 1:1 to 1:2.5, or from 1:1 to 1:1.5.
19. The composition according to a preceding implementation, wherein the composition comprises water and the crosslinked polymer is present in a concentration from 1-20 wt. %, from 1-10 wt. %, from 1-5 wt. %, from 5-10 wt. %, from 5-15 wt. %, from 10-15 wt. %, or from 10-20 wt. %.
20. The composition according to a preceding implementation, comprising at least one therapeutic agent.
21. The composition according to a preceding implementation, comprising a protein or nucleic acid.
22. A method of delivering a therapeutic agent to a subject in need thereof, comprising administering to the subject the composition according to any preceding implementation.
23. The method according to a preceding implementation, wherein the composition is administered subcutaneously, intramuscularly.
24. The method according to any preceding implementation, wherein the therapeutic agent comprises a biologic therapeutic agent or an antigen.
25. A method of vaccinating a subject in need thereof, comprising administering to the subject the composition according to any preceding implementation, wherein the composition comprises an antigen.
26. A method of making the composition according to a preceding implementation, comprising combining an a 7-oxanorbornene functionalized polymer and a multi-valent nucleophile-terminated compound in an aqueous composition.
27. The method according to a preceding implementation, wherein the aqueous composition further comprises a therapeutic agent.
28. The method according to a preceding implementation, wherein a solution comprising 7-oxanorbornene functionalized polymer is mixed with a solution comprising multi-valent nucleophile-terminated compound using a syringe-to-syringe process.
29. A method of delivering a therapeutic agent to a subject in need thereof, comprising administering to the subject the composition according to any preceding implementation, wherein the therapeutic agent is continuously released for a period of at least 14 days.

1. A composition comprising a crosslinked network obtained by crosslinking a mixture comprising a multi-valent nucleophile-terminated compound and a first polymer functionalized with oxanorbornene groups of Formula (1): 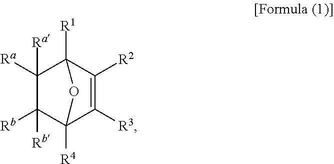
wherein:
R^{a} and R^{b} together form an epoxide or double bond;
R¹ is selected from H, halo, C₁₋₄alkyl, C₁₋₄haloalkyl, C₀₋₄alkC₆₋₁₂aryl, C₀₋₄alkC₃₋₁₂heteroaryl, and -L-polymer;
R⁴ is selected from H, halo, C₁₋₄alkyl, C₁₋₄haloalkyl, C₀₋₄alkC₆₋₁₂aryl, C₀₋₄alkC₃₋₁₂heteroaryl, and -L-polymer;
R^{a′} is selected from H, halo, C₁₋₄alkyl, C₁₋₄haloalkyl, C₀₋₄alkC₆₋₁₂aryl, C₀₋₄alkC₃₋₁₂heteroaryl, and -L-polymer;
R^{b′} is selected from H, halo, C₁₋₄alkyl, C₁₋₄haloalkyl, C₀₋₄alkC₆₋₁₂aryl, C₀₋₄alkC₃₋₁₂heteroaryl, and -L-polymer;
R² is selected from trifluoromethyl, cyano, nitro, C(═O)R^{c}, C(═O)OR^{c}, C(═O)NHR^{c}, and C(═O)N(R^{c})₂;
R³ is selected from trifluoromethyl, cyano, nitro, C(═O)R^{d}, C(═O)OR^{d}, C(═O)NHR^{d}, and C(═O)N(R^{d})₂;
R^{c} and R^{d} are independently a C₁₋₄alkyl group, C₀₋₄alkC₆₋₁₂aryl, or -L-polymer, or R^{c} and R^{d} together form a ring;
L is a linker; and
the oxanorbornene is substituted by a single -L-polymer group.

2. The compound of claim 1, wherein the oxanorbornene of Formula (1) is:

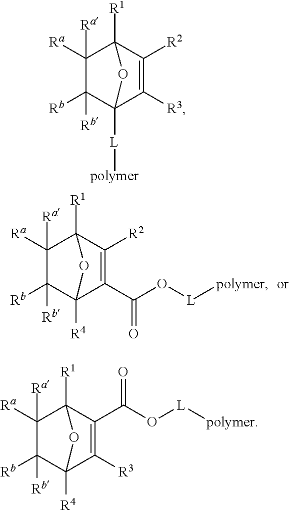

3. The composition of claim 1, wherein the oxanorbornene of Formula (1) has the Formula (a) or Formula (1e):

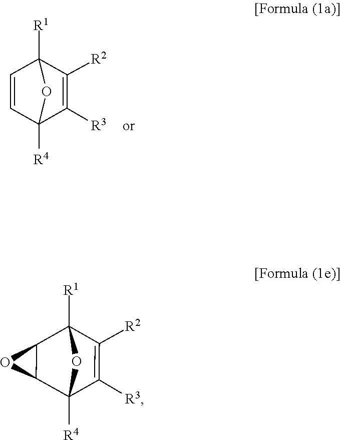

4. The composition of claim 1, wherein the oxanorbornene of Formula (1) is:

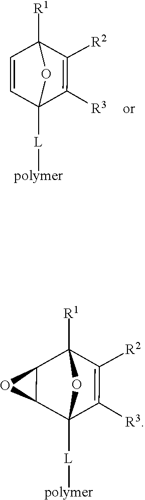

5. The composition of claim 1, wherein for the oxanorbornene of Formula (1):
a) L has the formula —[CH₂]ₓ—X¹—C₀₋₄alk-L*-C₀₋₄alk-X²—, wherein —[CH₂]ₓ— is bonded to the oxanorbornene and x is 0, 1, or 2; X¹ is null, NH, O, OC(═O), NHC(═O), or NHC(═O)NH; L* is null, C₁₋₆alkyl, C₁₋₆heteroalkyl, C₆₋₁₂aryl, C₃₋₁₂heteroaryl, C₃₋₁₂cycloalkyl, C₁₋₁₂heterocyclyl, or (CH₂CH₂O)_{z}, wherein z is 1-500, 1-5, 1-10, 5-15, 5-25, 10-50, 10-100, 25-50, 50-100, or 100-500; wherein said L* may be substituted one or more times by halo or hydroxyl; and X² is null, NH, O, C(═O), OC(═O), NHC(═O), NHC(═O)NH;
b) L is a (CH₂CH₂O)_{z}, wherein z is 1-500, 1-5, 1-10, 5-15, 5-25, 10-50, 10-100, 25-50, 50-100, or 100-500; or a peptide; or
c) L is —CH₂-L*-C₀₋₄alk-X²— or —CH₂—NHC(═O)—C₀₋₄alk-L*-C₀₋₄alk-X²—, wherein L* is C₁₋₆alkyl or C₃₋₁₂heteroaryl.

6-7. (canceled)

8. The composition of claim 5, wherein for the oxanorbornene of Formula (1), L* comprises the product of a click-cycloaddition.

9. (canceled)

10. The composition of claim 1, wherein for the oxanorbornene of Formula (1), L-polymer is: 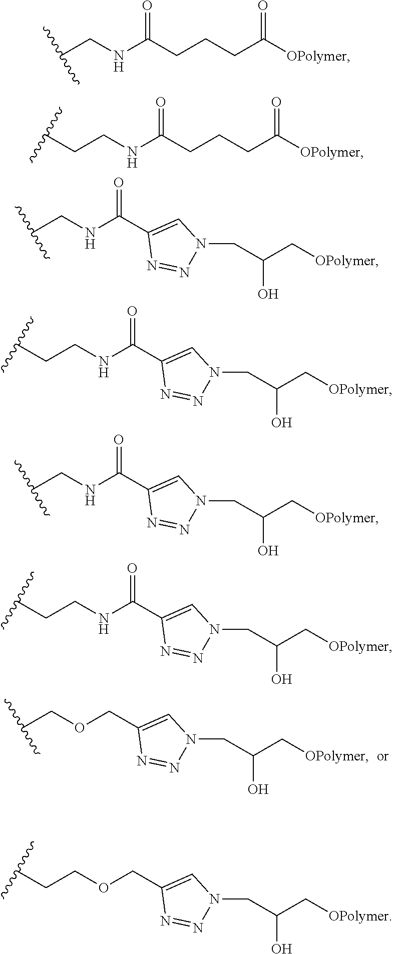
wherein the wavy line indicates the bond to the oxanorbornene, and a is 0, 1, 2, 3, 4, or 5.

11. The compound of claim 1, wherein for the oxanorbornene of Formula (1):
a) R² is CF₃ and R³ is CO₂Et, CO₂Me, or CO₂CH₂Ph; or R³ is CF₃ and R² is CO₂Et, CO₂Me, or CO₂CH₂Ph; or
b) R² is CO₂(CH₂)_{b}CO₂Polymer or C(═O)NH(CH₂)_{b}CO₂Polymer, and R³ is CF₃, CO₂Et, CO₂Me, or CO₂CH₂Ph; or R³ is CO₂(CH₂)_{b}CO₂Polymer or C(═O)NH(CH₂)_{b}CO₂Polymer, and R² is CF₃, CO₂Et, CO₂Me, or CO₂CH₂Ph; wherein b is 2, 3, 4, 5, 6, or 7.

12. (canceled)

13. The composition of claim 1, wherein for the oxanorbornene of Formula (1), R¹ is methyl.

14-19. (canceled)

20. The composition of claim 1, wherein the first polymer has a Mₙ from 5 kDa to 3,000 kDa and/or comprises oxanorbornene groups of Formula (1) in an amount (per monomer unit) from 0.5-100%.

21-24. (canceled)

25. The composition of claim 1, wherein the multi-valent nucleophile terminated compound has the formula: 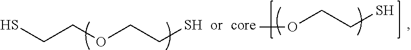
wherein the core has the formula: 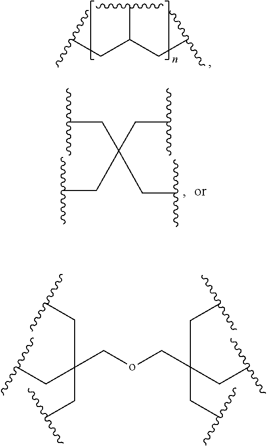
wherein n is an integer from 1-6; and
wherein the multi-valent nucleophile terminated compound has an average MW from 200-50,000 Da; and/or wherein the first polymer comprising oxanorbornene groups and the multi-valent nucleophile-terminated compound are combined in a weight ratio from 10:1 to 1:10.

26. (canceled)

27. The composition of claim 1, wherein the mixture further comprises a second polymer functionalized with oxanorbornene groups of Formula (2): 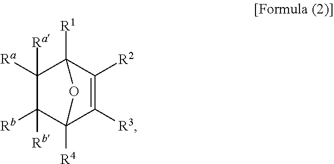
wherein:
R^{a} and R^{b} together form an epoxide or double bond;
R¹ is selected from H, halo, C₁₋₄alkyl, C₁₋₄haloalkyl, C₀₋₄alkC₆₋₁₂aryl, C₀₋₄alkC₃₋₁₂heteroaryl, and -L-polymer;
R⁴ is selected from H, halo, C₁₋₄alkyl, C₁₋₄haloalkyl, C₀₋₄alkC₆₋₁₂aryl, C₀₋₄alkC₃₋₁₂heteroaryl, and -L-polymer;
R^{a′} is selected from H, halo, C₁₋₄alkyl, C₁₋₄haloalkyl, C₀₋₄alkC₆₋₁₂aryl, C₀₋₄alkC₃₋₁₂heteroaryl, and -L-polymer;
R^{b′} is selected from H, halo, C₁₋₄alkyl, C₁₋₄haloalkyl, C₀₋₄alkC₆₋₁₂aryl, C₀₋₄alkC₃₋₁₂heteroaryl, and -L-polymer;
R² is selected from trifluoromethyl, cyano, nitro, C(═O)R^{c}, C(═O)OR^{c}, C(═O)NHR^{c}, and C(═O)N(R^{c})₂;
R³ is selected from trifluoromethyl, cyano, nitro, C(═O)R^{d}, C(═O)OR^{d}, C(═O)NHR^{d}, and C(═O)N(R^{d})₂;
R^{c} and R^{d} are independently a C₁₋₄alkyl group, C₀₋₄alkC₆₋₁₂aryl, or -L-polymer, or R^{c} and R^{d} together form a ring;
L is a linker;
the oxanorbornene is substituted by a single -L-polymer group; and
the first polymer functionalized with oxanorbornene groups is not the same as the second polymer functionalized with oxanorbornene groups.

28-47. (canceled)

48. The composition of claim 1, wherein the composition comprises water and the crosslinked network is present in a concentration from 1-50 wt. %.

49. (canceled)

50. The composition of claim 1, further comprising a cargo that is non-covalently entrapped in the crosslinked network or a cargo that is covalently linked to an oxanorbornene group.

51. (canceled)

52. The composition of claim 1, wherein the mixture further comprises a third polymer functionalized with cargo-linked oxanorbornene of Formula (3) 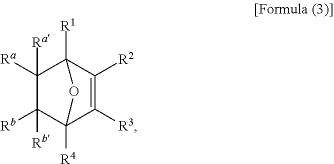
wherein:
R^{a} and R^{b} together form an epoxide or double bond;
R¹ is selected from H, halo, C₁₋₄alkyl, C₁₋₄haloalkyl, C₀₋₄alkC₆₋₁₂aryl, C₀₋₄alkC₃₋₁₂heteroaryl, -L-polymer, and Lt-cargo;
R⁴ is selected from H, halo, C₁₋₄alkyl, C₁₋₄haloalkyl, C₀₋₄alkC₆₋₁₂aryl, C₀₋₄alkC₃₋₁₂heteroaryl, -L-polymer, and Lt-cargo;
R^{a′} is selected from H, halo, C₁₋₄alkyl, C₁₋₄haloalkyl, C₀₋₄alkC₆₋₁₂aryl, C₀₋₄alkC₃₋₁₂heteroaryl, or -L-polymer, and Lt-cargo;
R^{b′} is selected from H, halo, C₁₋₄alkyl, C₁₋₄haloalkyl, C₀₋₄alkC₆₋₁₂aryl, C₀₋₄alkC₃₋₁₂heteroaryl, or -L-polymer, and Lt-cargo;
R² is selected from trifluoromethyl, cyano, nitro, C(═O)R^{c}, C(═O)OR^{c}, C(═O)NHR^{c}, and C(═O)N(R^{c})₂;
R³ is selected from trifluoromethyl, cyano, nitro, C(═O)R^{d}, C(═O)OR^{d}, C(═O)NHR^{d}, and C(═O)N(R^{d})₂;
R^{c} and R^{d} are independently a C₁₋₄alkyl group, C₀₋₄alkC₆₋₁₂aryl, -L-polymer, or Lt-cargo, or R^{c} and R^{d} together form a ring;
L is a linker;
L¹ is a linker; and
the oxanorbornene is substituted by a single -L-polymer group.

53-64. (canceled)

65. The composition of claim 52, wherein for the oxanorbornene of Formula (3), Lt-cargo has the formula —[CH₂]_{y}—C₀₋₄alk-X¹-L*-C₀₋₄alk-X²-cargo, wherein the —[CH₂]_{y}— is bonded to the oxanorbornene and y is 0, 1 or 2;
X¹ is null, NH, O, OC(═O), NHC(═O), or NHC(═O)NH;
L* is null, C₁₋₆alkyl, C₁₋₆heteroalkyl, C₆₋₁₂aryl, C₃₋₁₂heteroaryl, C₃₋₁₂cycloalkyl, C₁₋₁₂heterocyclyl, or (CH₂CH₂O)_{z}, wherein z is 1-500, 1-5, 1-10, 5-15, 5-25, 10-50, 10-100, 25-50, 50-100, or 100-500; wherein said L* may be substituted one or more times by halo or hydroxyl; and
X² is null, NH, O, C(═O), OC(═O), NHC(═O), NHC(═O)NH.

66-77. (canceled)

78. A method of delivering a therapeutic agent to a subject in need thereof, comprising administering to the subject the composition of claim 1, wherein the composition comprises at least one therapeutic agent.

79-80. (canceled)

81. A method of vaccinating a subject in need thereof, comprising administering to the subject the composition of claim 1, wherein the composition comprises an antigen, and adjuvant, or a combination thereof.

82. A method of making the composition of claim 1, comprising combining an a 7-oxanorbornene functionalized polymer and a multi-valent nucleophile-terminated compound in an aqueous composition.

83. The method according to claim 82, wherein the aqueous composition further comprises a therapeutic agent; and/or
wherein a solution comprising 7-oxanorbornene functionalized polymer is mixed with a solution comprising multi-valent nucleophile-terminated compound using a syringe-to-syringe process.

84-85. (canceled)
